(19) United States
(12) Patent Application Publication
(10) Pub. No.: US 2016/0051316 A1
(43) Pub. Date: Feb. 25, 2016

(54) ELECTROSURGICAL ELECTRODE MECHANISM

(71) Applicant: Ethicon Endo-Surgery, Inc., Cincinnati, OH (US)

(72) Inventor: Chad P. Boudreaux, Cincinnati, OH (US)

(21) Appl. No.: 14/467,990

(22) Filed: Aug. 25, 2014

(51) Int. Cl. A61B 18/14

(52) U.S. Cl. CPC A61B 18/1445; A61B 2018/00101

(57) ABSTRACT

An electrosurgical instrument comprises a shaft and end effector positioned distally of the shaft. A first jaw comprises a first jaw body, a first jaw energy delivery surface, a first jaw electrode positioned at the first jaw energy delivery surface, an insulator positioned between the first jaw electrode and the first jaw body to thermally insulate the first jaw electrode and the first jaw body, and an inner surface positioned at the first jaw energy delivery surface, thermally coupled to the first jaw body, and electrically coupled to the first jaw body. A second jaw pivotable towards the first jaw from an open position to a closed position comprises a second jaw energy delivery surface. The first jaw energy delivery surface and the second jaw energy delivery surface face one another when the end effector is in the closed position.

### CROSS-REFERENCE TO RELATED APPLICATIONS

This application is related to Application Docket No. END7521USNP/140426 titled “LOCKOUT DISABLING MECHANISM,” filed concurrently herewith and Application Docket No. END7522USNP/140427 titled “SIMULTANEOUS I-BEAM AND SPRING DRIVEN CAM JAW CLOSURE MECHANISM,” filed concurrently herewith; each of which is incorporated herein by reference in its entirety.

### INTRODUCTION

The present disclosure is related generally to electrosurgical devices with various mechanisms for clamping and treating tissue. In particular, the present disclosure is related to electrosurgical devices with electrode mechanisms configured for heat management.

Conventional electrosurgical devices comprise jaws with electrodes for treating tissue. At least one of the electrodes is thermally coupled to the remainder of its jaw, which causes heat to be wicked away from a tissue treatment area. If the heat flow away from the tissue treatment area is too high, then it may take inordinately long to complete treatment of the tissue. Accordingly, to provide improved heat management, the following disclosure describes various solutions for managing heat flow.

While several devices have been made and used, it is believed that no one prior to the inventors has made or used the embodiments described in the appended claims.

### SUMMARY

In one embodiment, an electrosurgical instrument is provided. The electrosurgical instrument comprises a shaft and an end effector positioned at a distal end of the shaft. The end effector comprises a first jaw and a second jaw. The first jaw comprises a first jaw body, a first jaw energy delivery surface, a first jaw electrode positioned at the first jaw energy delivery surface, an insulator positioned between the first jaw electrode and the first jaw body to thermally insulate the first jaw electrode and the first jaw body, and an inner surface positioned at the first jaw energy delivery surface, thermally coupled to the first jaw body, and electrically coupled to the first jaw body. A second jaw is pivotable towards the first jaw from an open position to a closed position. The second jaw comprises a second jaw energy delivery surface, and wherein the first jaw energy delivery surface and the second jaw energy delivery surface face one another when the end effector is in the closed position.

In another embodiment, the second jaw further comprises a second jaw body, a second jaw electrode at the second jaw energy delivery surface, and a second jaw insulator positioned between the second jaw electrode and the second jaw body to thermally insulate the second jaw electrode and the second jaw body.

In another embodiment, the first jaw comprises a positive temperature coefficient (PTC) element at the first jaw energy delivery surface.

In another embodiment, the first jaw electrode and the PTC component face the second jaw electrode, wherein the inner surface comprises a tooth having a surface opposite at least a portion of the second jaw electrode, and wherein a width of the second jaw electrode is about equal to a sum of: a width of the first jaw electrode; a width of the at least one surface of the tooth; and a width of the PTC component. In another embodiment, the width of the first jaw electrode is between about 39% and about 45% of the width of the second jaw electrode. In another embodiment, the width of the PTC component is between about 33% and 39% of the width of the second jaw electrode. In another embodiment, the width of the at least one surface is between about 19% and 25% of the width of the second jaw electrode.

In another embodiment, the first jaw electrode faces the second jaw electrode, wherein the inner surface comprises a tooth with at least one surface opposite at least a portion of the second jaw electrode, and wherein a width of the second jaw electrode is about equal to a sum of: a width of the first jaw electrode and a width of the at least one surface of the inner surface.

In another embodiment, the first jaw electrode is coupled to the first jaw body at least one connection point, wherein the at least one connection point is positioned at a proximal portion of the first jaw body.

In another embodiment, the first jaw defines a first jaw channel, the second jaw defines a second jaw channel, and further comprising a cutting element extendable distally through the first and second jaw channels to transition the end effector to the closed position.

In another embodiment, the inner surface is positioned adjacent the first jaw channel, wherein the inner surface comprises a plurality of teeth, and wherein a first portion of the plurality of teeth are positioned on a first side of the first jaw channel and a second portion of the plurality of teeth are positioned on a second side of the first jaw channel. In another embodiment, the inner surface comprises a plurality of teeth positioned at a regular interval along a length of the first jaw. In another embodiment, the inner surface further comprises inter-teeth recesses positioned between the plurality of teeth.

In one embodiment, an electrosurgical instrument is provided. The electrosurgical instrument comprises a shaft and an end effector positioned at a distal end of the shaft. The end effector comprises a first jaw and a second jaw. The first jaw comprises a first jaw body and a first jaw energy delivery surface. The second jaw is pivotable towards the first jaw from an open position to a closed position. The second jaw comprises a second jaw body, a second jaw energy delivery surface. The first jaw energy delivery surface and the second jaw energy delivery surface face one another when the end effector is in the closed position. A second jaw electrode is positioned at the second jaw energy delivery surface. A second jaw inner surface is positioned at the second jaw energy delivery surface and thermally connected to the second jaw body. A second jaw insulator is positioned between the second jaw heat conductor and the second jaw body.

In another embodiment, the inner surface comprises a plurality of teeth.

In another embodiment, the first jaw defines a first jaw channel, the second jaw defines a second jaw channel, and further comprising a cutting element extendable distally through the first and second jaw channels to transition the end effector to the closed position.

In another embodiment, the plurality of teeth comprises a first tooth on a first side of the second jaw channel and a second tooth on a second side of the second jaw channel opposite the first heat conductor.

In another embodiment, the first jaw further comprises a first jaw electrode positioned at the first jaw energy delivery surface and an insulator positioned between the first jaw electrode and the first jaw body to thermally insulate the first jaw electrode and the first jaw body.

In another embodiment, the first jaw further comprises a first jaw inner surface comprising a plurality of teeth and a plurality of inter-teeth recesses positioned between the plurality of teeth, and wherein when the end effector is in the closed position, the second jaw heat conductor aligns with at least one of the inter-teeth recesses.

In another embodiment, the second jaw insulator is also positioned between the second jaw electrode and the second jaw body to thermally insulate the second jaw electrode from the second jaw body.

The foregoing summary is illustrative only and is not intended to be in any way limiting. In addition to the illustrative aspects, embodiments, and features described above, further aspects, embodiments, and features will become apparent by reference to the drawings and the following detailed description.

### FIGURES

The novel features of the embodiments described herein are set forth with particularity in the appended claims. The embodiments, however, both as to organization and methods of operation may be better understood by reference to the following description, taken in conjunction with the accompanying drawings as follows.

FIG. 1 illustrates a surgical instrument comprising a knife lockout disabling mechanism, according to one embodiment.

FIG. 2 is a perspective view of a handle assembly of the surgical instrument illustrated in FIG. 1 with the left handle housing shroud and several sheaths in the shaft assembly removed, according to one embodiment.

FIG. 3 illustrates a perspective view of an end effector for use with an electrosurgical instrument, according to one embodiment.

FIG. 4 illustrates a perspective view of an end effector for use with an electrosurgical instrument, according to one embodiment.

FIG. 5 illustrates perspective cross-sectional view of the end effector of FIGS. 3 and 4 in a closed configuration, according to one embodiment.

FIG. 6 illustrates a cross-sectional view of the end effector of FIGS. 3 and 4 in the closed configuration, according to one embodiment.

FIG. 7 illustrates a perspective view of the upper jaw of FIGS. 3-6 showing additional features of the electrode and heat barrier, according to one embodiment.

FIG. 8 illustrates a cross-sectional view of the end effector of FIGS. 3 and 4 in the closed configuration illustrating current and heat flows, according to one embodiment.

FIG. 9 illustrates a cross-sectional view of the end effector of FIGS. 3 and 4 in the closed configuration showing example dimensions for various components of the end effector, according to one embodiment.

FIG. 9A illustrates a cross-sectional view of the end effector in the closed configuration showing an alternate upper electrode, according to one embodiment.

FIG. 10 illustrates the lower jaw comprising an inner surface with conductive teeth, according to one embodiment.

FIG. 11 illustrates an exploded view of the lower jaw of FIG. 10, according to one embodiment.

FIG. 12 illustrates a cross-sectional view of the lower jaw of FIG. 10 taken at a set of the conductive teeth, according to one embodiment.

FIG. 13 is a side elevation view of a handle assembly of a surgical instrument, similar to the surgical instrument shown in FIGS. 1 and 2, with the left handle housing shroud removed, and without the lockout disabling mechanism, according to one embodiment.

FIG. 14 is an exploded view of the shaft assembly, end effector, yoke, and rack portions of the surgical instrument shown in FIGS. 1 and 2, according to one embodiment.

FIG. 15 is a perspective view of the shaft assembly, end effector, yoke, and rack shown in FIG. 14 in the assembled state, according to one embodiment.

FIG. 16 is a perspective view of the shaft assembly, end effector, yoke, and rack shown in FIG. 15, according to one embodiment, with the electrically insulative nonconductive tube removed to show the functional components of the shaft assembly in the assembled state.

FIG. 17 is a sectional view taken along a longitudinal axis of the shaft assembly, yoke, and rack shown in FIG. 15, according to one embodiment, to show the functional components of the shaft assembly in the assembled state.

FIG. 18 is partial perspective view of the shaft assembly shown in FIG. 17, according to one embodiment.

FIG. 19 is a side view of an end effector portion of the surgical instrument shown in FIGS. 1 and 2 with the jaws open, according to one embodiment.

FIG. 20 shows the closure bar and I-beam member at the initial stage of clamp closure and firing sequence where the I-beam member is located at the base of a ramp in the upper jaw, according to one embodiment.

FIG. 21 shows the closure bar and I-beam member further advanced distally than shown in FIG. 20, where the I-beam member is located at an intermediate position along the ramp in the upper jaw, according to one embodiment.

FIG. 22 shows the closure bar and I-beam member further advanced distally than shown in FIG. 21 where the I-beam member is located at the top of the ramp in the upper jaw, according to one embodiment.

FIG. 23 shows the closure bar and I-beam member further advanced distally than shown in FIG. 22, where the I-beam member is located past the ramp in the upper jaw, according to one embodiment.

FIG. 24 is a side elevational view of the surgical instrument shown in FIGS. 1 and 2 with the left housing shroud removed, shaft assembly sheaths removed, and the jaw fully open, according to one embodiment.

FIG. 25 is a perspective view of the surgical instrument shown in FIG. 24 with the right housing shroud removed, according to one embodiment.

FIG. 26 is another perspective view of the surgical instrument shown in FIG. 25, according to one embodiment.

FIG. 27 is a side elevational view of the surgical instrument shown in FIG. 24 with the right housing shroud removed, according to one embodiment.

FIG. 28 is a side elevational view of the surgical instrument shown in FIG. 24 with the firing plate removed, according to one embodiment.

FIG. 29 is a side elevational view of the surgical instrument shown in FIG. 28 with the lockout defeat mechanism slider removed, according to one embodiment.

FIG. 30 is a side elevational view of the surgical instrument shown in FIG. 28 with the toggle clamp and yoke removed, according to one embodiment.

FIG. 31 is a partial perspective view of the surgical instrument shown in FIG. 30, according to one embodiment.

FIG. 32 is a partial perspective view of the surgical instrument shown in FIG. 31 with the firing plate replaced, according to one embodiment.

FIG. 33 is a partial perspective view of the surgical instrument shown in FIG. 32 with the lockout defeat mechanism slider, lever arm, and lock arm removed, according to one embodiment.

FIG. 34 is a side elevational view of the surgical instrument shown in FIGS. 1 and 2 with the left and right housing shrouds removed, shaft assembly sheaths removed, jaws clamped, and the lockout defeat mechanism enabled, e.g., in the “ON” position, according to one embodiment.

FIG. 35 is a side elevational view of the surgical instrument shown in FIGS. 1 and 2 with the left and right housing shrouds removed, shaft assembly sheaths removed, jaws fully closed, knife fully fired, and the lockout defeat mechanism disabled, e.g., in the “OFF” position, according to one embodiment.

FIG. 36 is a side elevational view of the surgical instrument shown in FIGS. 1 and 2 with the left and right housing shrouds removed, shaft assembly sheaths removed, jaws fully open, knife not fired, and the lockout defeat mechanism disabled, e.g., in the “OFF” position, according to one embodiment.

### DESCRIPTION

In the following detailed description, reference is made to the accompanying drawings, which form a part hereof. In the drawings, similar symbols and reference characters typically identify similar components throughout the several views, unless context dictates otherwise. The illustrative embodiments described in the detailed description, drawings, and claims are not meant to be limiting. Other embodiments may be utilized, and other changes may be made, without departing from the scope of the subject matter presented here.

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

Before explaining the various embodiments of the surgical devices having a knife lockout disabling mechanism in detail, it should be noted that the various embodiments disclosed herein are not limited in their application or use to the details of construction and arrangement of parts illustrated in the accompanying drawings and description. Rather, the disclosed embodiments may be positioned or incorporated in other embodiments, variations and modifications thereof, and may be practiced or carried out in various ways. Accordingly, embodiments of the surgical devices with two-stage triggers disclosed herein are illustrative in nature and are not meant to limit the scope or application thereof. Furthermore, unless otherwise indicated, the terms and expressions employed herein have been chosen for the purpose of describing the embodiments for the convenience of the reader and are not to limit the scope thereof. In addition, it should be understood that any one or more of the disclosed embodiments, expressions of embodiments, and/or examples thereof, can be combined with any one or more of the other disclosed embodiments, expressions of embodiments, and/or examples thereof, without limitation.

Also, in the following description, it is to be understood that terms such as front, back, inside, outside, top, bottom and the like are words of convenience and are not to be construed as limiting terms. Terminology used herein is not meant to be limiting insofar as devices described herein, or portions thereof, may be attached or utilized in other orientations. The various embodiments will be described in more detail with reference to the drawings.

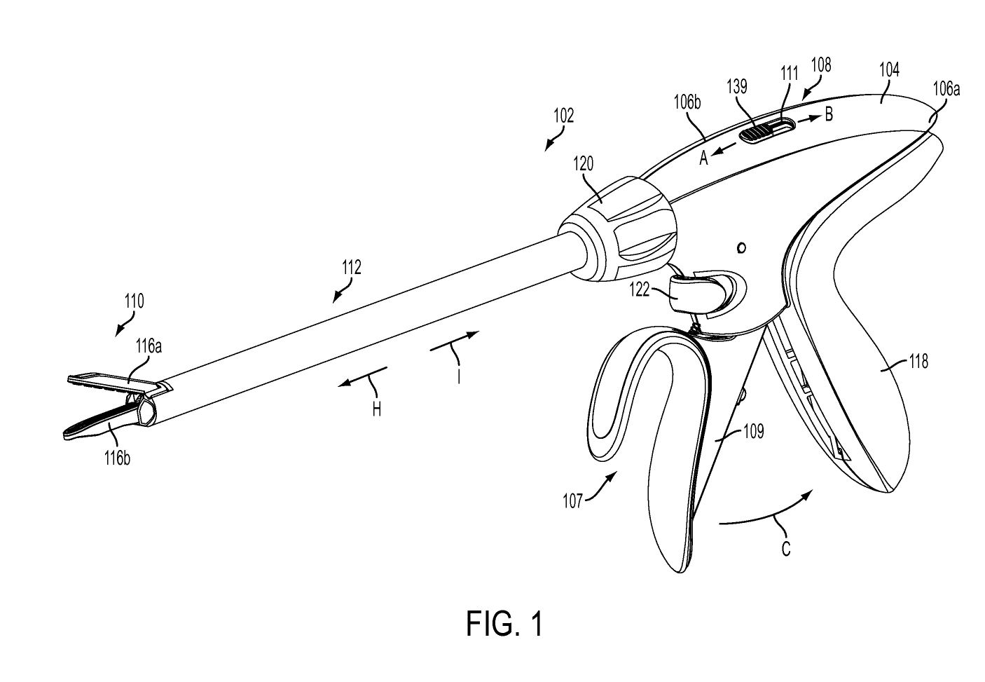

Turning now to the figures, FIG. 1 illustrates a surgical instrument 102 comprising a trigger assembly 107 and a closure system arrangement for closing the jaws 110 comprising a separate spring driven cam closure mechanism that is independent of the I-beam closure mechanism. The spring driven cam closure system and the I-beam closure system are configured to independently close a set of opposing jaws 116a, 116b, and independently fire a cutting element in the end effector 110. The trigger assembly 107 is configured to clamp and independently fire an end effector 110 coupled to the shaft assembly 112 of the surgical instrument 102. In the embodiment shown in FIG. 1, the surgical instrument comprises a trigger assembly 107 and a lockout disabling mechanism 108. In this view, a first jaw member 116a of an end effector 110 is fully open and the knife lockout disabling mechanism 108 is located in the off position. The knife lockout disabling mechanism 108 is configured to clamp and fire an end effector 110 coupled to the surgical instrument 102. The surgical instrument 102 comprises a handle assembly 104, a shaft assembly 112, and the end effector 110. The shaft assembly 112 comprises a proximal end and a distal end. The proximal end of the shaft assembly 112 is coupled to the distal end of the handle assembly 104. The end effector 110 is coupled to the distal end of the shaft assembly 112. The handle assembly 104 comprises a pistol grip 118. The handle assembly 104 comprises a left handle housing shroud 106a and a right handle housing shroud 106b. The trigger assembly 107 comprises a trigger 109 actuatable towards the pistol grip 118. The knife lockout disabling mechanism 108 comprises a button 139, or knob, that is actuatable for adjusting or controlling the position of the knife lockout disabling mechanism 108 between first and second positions A and B (A=Distal and B=Proximal relative to the clinician) within a slot 111 formed in the left handle housing shroud 106a. A rotatable shaft knob 120 is configured to rotate the shaft assembly 112 with respect to the handle assembly 104. The handle assembly 104 further comprises an energy button 122 configured to provide electrosurgical energy to one or more electrodes in the end effector 110.

The knife lockout mechanism forces the user to first clamp (close the jaws 110), energize the electrodes, then cut the tissue (fire the knife). The knife unlock feature contains the energy button 122 so that the energy button 122 has to be depressed before the knife can be released or that the single trigger can move the rack 136 forward. The single trigger 109 closes the jaws in the first ˜13 degrees of stroke. The single trigger 109 fires the knife in the last ˜29 degrees of stroke. The lockout is the stop in between the first stroke and the second stroke. An energy switch (not shown) is located underneath the energy button 122 housing. Accordingly, the lock release mechanism also is the energy delivery element.

The shaft assembly 112 comprises a closure/jaw actuator, a firing/cutting member actuator, and an outer sheath. In some embodiments, the outer sheath comprises the closure actuator. The outer sheath comprises one or more contact electrodes on a distal end configured to interface with the end effector 110. The one or more contact electrodes are operatively coupled to the energy button 122 and an energy source (not shown).

The energy source may be suitable for therapeutic tissue treatment, tissue cauterization/sealing, as well as sub-therapeutic treatment and measurement. The energy button 122 controls the delivery of energy to the electrodes. As used throughout this disclosure, a button refers to a switch mechanism for controlling some aspect of a machine or a process. The buttons may be made out of a hard material such as usually plastic or metal. The surface may be formed or shaped to accommodate the human finger or hand, so as to be easily depressed or pushed. Buttons can be most often biased switches, even though many un-biased buttons (due to their physical nature) require a spring to return to their un-pushed state. Terms for the “pushing” of the button, may include press, depress, mash, and punch.

In some embodiments, an end effector 110 is coupled to the distal end of the shaft assembly 112. The end effector 110 comprises a first jaw member 116a and a second jaw member 116b. The first jaw member 116a is pivotally coupled to the second jaw member 116b. The first jaw member 116a is pivotally moveable with respect to the second jaw member 116b to grasp tissue therebetween. In some embodiments, the second jaw member 116b is fixed. In other embodiments, the first jaw member 116a and the second jaw member 116b are pivotally movable. The end effector 110 comprises at least one electrode. The electrode is configured to deliver energy. Energy delivered by the electrode may comprise, for example, radiofrequency (RF) energy, sub-therapeutic RF energy, ultrasonic energy, and/or other suitable forms of energy. In some embodiments, a cutting member (not shown) is receivable within a longitudinal slot defined by the first jaw member 116a and/or the second jaw member 116b. The cutting member is configured to cut tissue grasped between the first jaw member 116a and the second jaw member 116b. In some embodiments, the cutting member comprises an electrode for delivering energy, such as, for example, RF and/or ultrasonic energy.

In certain instances, as described above, the surgical instrument 102 may include an automatic energy lockout mechanism. The energy lockout mechanism can be associated with a closure mechanism of the surgical instrument 102. In certain instances, the energy lockout mechanism can be configured to permit energy delivery to the end effector 10 when the energy delivery button 122 is actuated if the jaw members 116a and 116b are in an open configuration. In certain instances, the energy lockout mechanism may be configured to deny energy delivery to the end effector 110 when the energy delivery button 122 is actuated if the jaw members 116a and 116b are in a closed configuration. In certain instances, the energy lockout mechanism automatically transitions from permitting the energy delivery to denying the energy delivery when the jaw members 116a and 116b are transitioned from the closed configuration to the open configuration, for example. In certain instances, the energy lockout mechanism automatically transitions from denying the energy delivery to permitting the energy delivery when the jaw members 116a and 116b are transitioned from the open configuration to the closed configuration, for example.

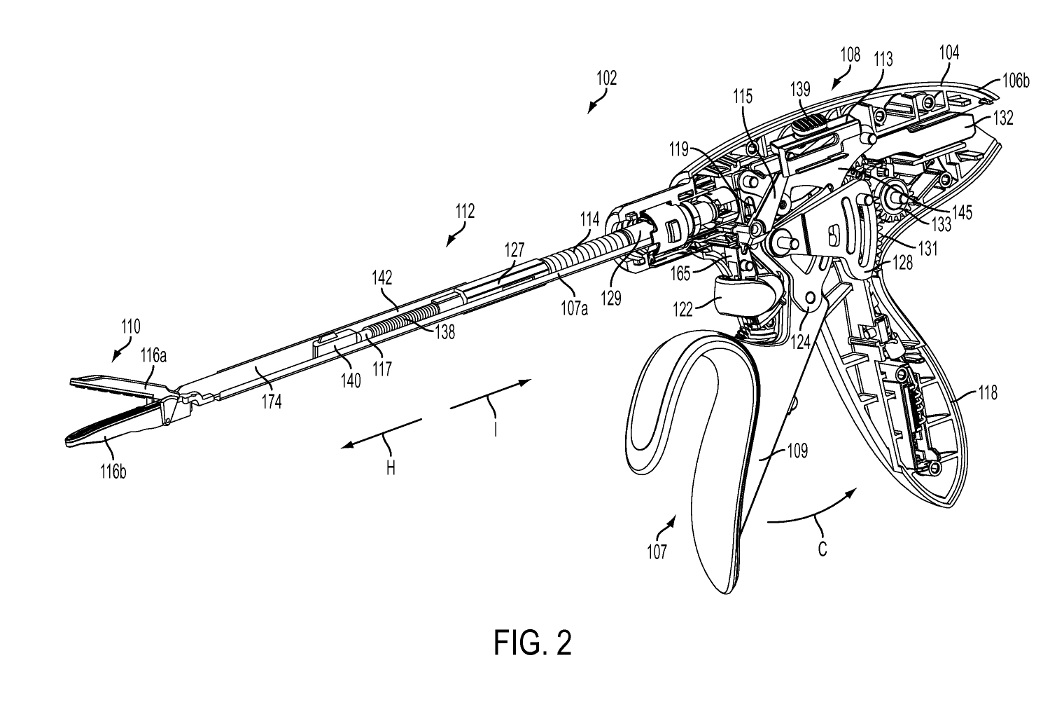

FIG. 2 is a perspective view of a handle assembly 104 of a surgical instrument 102 illustrated in FIG. 1, according to one embodiment, with the right housing shroud 106a and the outer and inner sheaths of the shaft assembly 112 removed to show some of the internal mechanisms. The left handle housing shroud 106b of the handle assembly 104 comprises the knife lockout disabling mechanism 108. The button 139 is located in the first “off” position A (A=distal relative to the clinician) within the slot 111 formed in the right handle housing shroud 106a. In the illustrated embodiment, position B (B=proximal relative to the clinician) corresponds to the second “on” position of the knife lockout disabling mechanism 108, where the knife lockout mechanism remains disabled until the button is switched back to position A. Accordingly, position A corresponds to the enabled state of the knife lockout mechanism and position B corresponds to the disabled state of the knife lockout mechanism. Stated differently, position A corresponds to the “off” state of the knife lockout disabling mechanism 108 and position B corresponds to the “on” state of the knife lockout disabling mechanism 108. When the knife lockout mechanism is in the disabled state, the energy button 122 may appear to be depressed to provide a visual indication to the clinician that the knife lockout mechanism has been disabled but without energizing the electrodes in the end effector 110 (FIG. 1). When the knife lockout mechanism is disabled, the knife may be fired at will without the need to apply electrosurgical energy to one or more electrodes in the end effector 110.

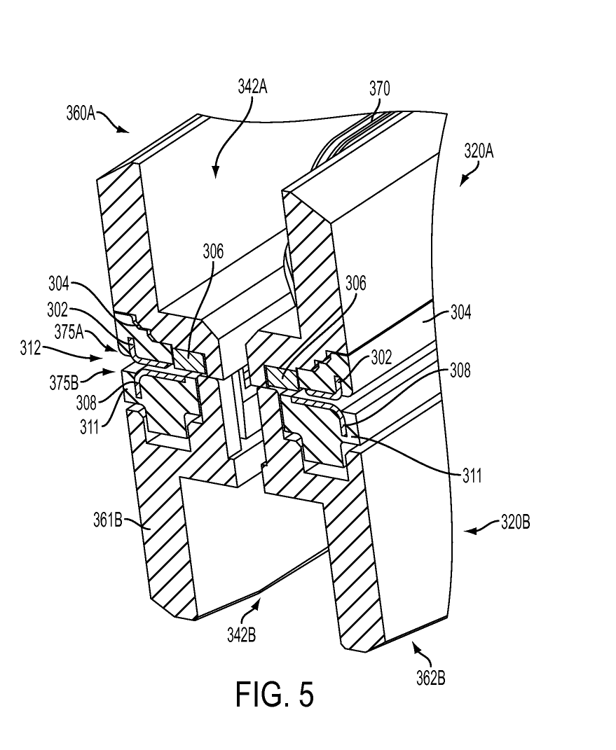

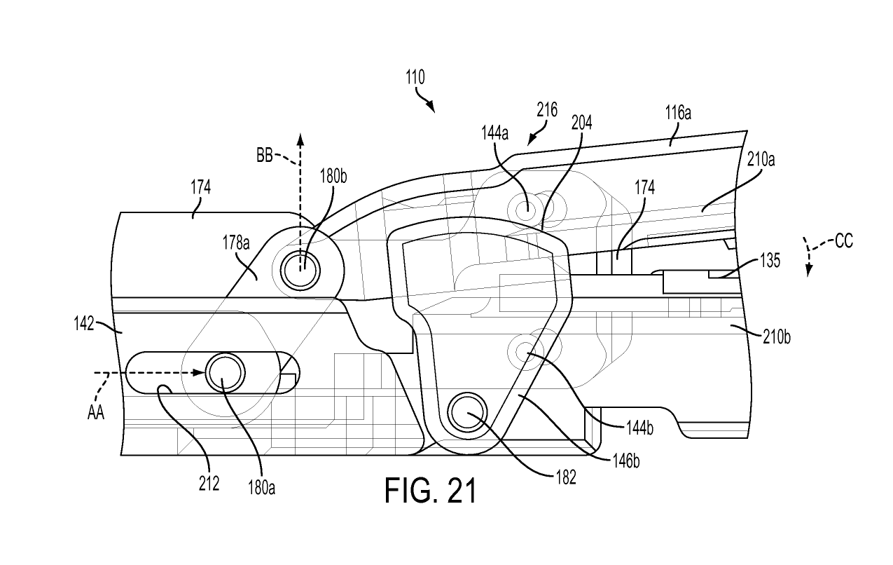

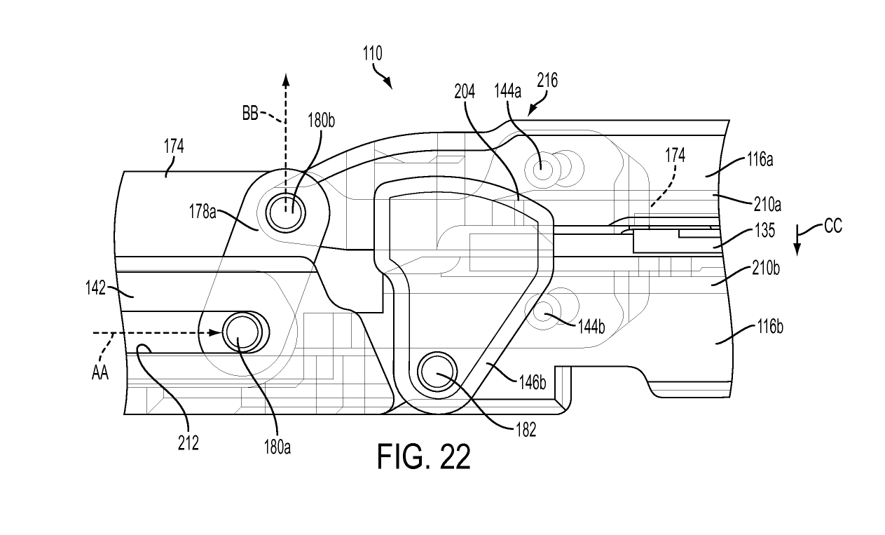

The trigger assembly 107 comprises the necessary components for closing the jaw members 116a, 116b and firing the cutting member or knife bands 142. The trigger assembly 107 comprises a trigger plate 124 and firing plate 128 operatively coupled to the trigger 109. Squeezing the trigger 109 in direction C towards the pistol grip 118 rotates the trigger plate 124 which operates the toggle clamp 145 to advance a yoke 132 and a closure actuator 123 distally to close the jaw members 116a, 116b of the end effector. Initial rotation of the trigger plate 124 also slightly rotates the firing plate 128. The firing plate 128 comprises a sector gear with a plurality of teeth 131 that engage and rotate a first pinion gear 133, which engages a second pinion gear 134 to advance a rack 136 (neither is shown in this view). A lock arm 157 (shown in FIGS. 21-22, for example) is operatively coupled to a lever arm 115, an unlock arm 119, and a lockout element 165. When the instrument 102 is in normal lockout mode, the lock arm 157 engages a notch 158 (shown in FIGS. 4 and 21-23, for example) in the rack 136 to lock the rack 136 and prevent the rack 136 from moving distally (firing) no matter how hard the trigger 109 is squeezed.

The single trigger 109 closes the jaws in the first ˜13 degrees of stroke. The trigger plate 24 is configured to interface with the trigger plate 124 during rotation of the trigger 109 from an initial position to a first rotation, which is ˜13 degrees of stroke, for example. The trigger plate 124 is operably coupled to the firing plate 128. In certain instances, the firing plate 128 may include a first slot 128a and a second slot 128b. The first slot 128a receives a drive pin 148 fixedly coupled to the trigger plate 124. The pin 148 slidably moves within the first slot 128a. Rotation of the trigger plate 124, while the pin 148 is slidably received within the first slot 128a, drives rotation of the firing plate 128. The teeth 131 of the sector gear engage and rotate the first pinion 133, which in turn drives the second pinion 134, which drives the rack 136 distally to fire the cutting element, or knife, but only when the knife lockout is unlocked, released, or disabled.

The single trigger 109 fires the knife in the last ˜29 degrees of stroke. Rotation of the trigger plate 124 beyond a predetermined rotation such as, for example, the first rotation, causes rotation of the firing plate 128. Rotation of the firing plate 128 deploys a cutting member within the end effector 110. For example, in the illustrated embodiment, the firing plate 128 comprises a sector gear operably coupled to a rack 136 through the first and second pinions 133, 134. The firing plate 128 comprises a plurality of teeth 131 configured to interface with the first pinion 133. Rotation of the firing plate 128 rotates the first and second pinions 133, 134, to drive the rack 136 distally. Distal movement of the rack 136 drives the cutting member actuator distally, causing deployment of the cutting member (e.g., knife) within the end effector 110.

The lockout is the stop in between the first stroke and the second stroke. Turning back now to the description of the lockout disabling mechanism 108, when the slider 113 button 139 portion is in located in position A, the lock arm 157 cam be released by pressing or actuating the energy button 122 to rotate the lockout element 165, which rotates the unlock arm 119 to release the lock arm 157. Once the lock arm 157 is released, the rack 136 is enabled to advance distally and fire the knife by squeezing the trigger 109 in direction C further towards the pistol grip 118. As the trigger 109 is squeezed, the firing plate 128 rotates and drives the first pinion gear 133, which drives the second pinion gear 134 to drive the rack 136.

When the button 139 is located in position B, the slider 113 rotates the lever arm 115, which rotates the unlock arm 119 to releases the lock arm 157. While the button 139 is in position B, the rack 136 can be fired without the need to press energy button 122 to rotate the lockout element 165. A detent may be provided to hold the button in either position A or B. These and other features are described in more detail hereinbelow.

The shaft assembly 112 comprises a closure/jaw actuator and a firing/cutting member actuator. The closure/jaw actuator comprises a yoke 132 and toggle clamp 145 assembly operatively coupled to a closure actuator 112 which acts on a closure spring 114 coupled to a spring-to-bar interface element 127 and a closure bar 116. In one instance the closure bar 116 is operatively coupled to the jaw members 116a, 116b via at least one linkage. The firing/cutting member actuator comprises a rack 136 operatively coupled to a firing bar 117, which is slidably received within the closure actuator 112 and the closure spring 114. The firing bar 117 is coupled to a knife pusher block 140 and a flexible I-beam knife band 142 comprising multiple flexible bands fastened together and a cutting element at the distal end. Advancing the rack 136 in the distal direction advances the cutting element band 142 distally through a channel or slot formed in the jaw members 116a, 116b.

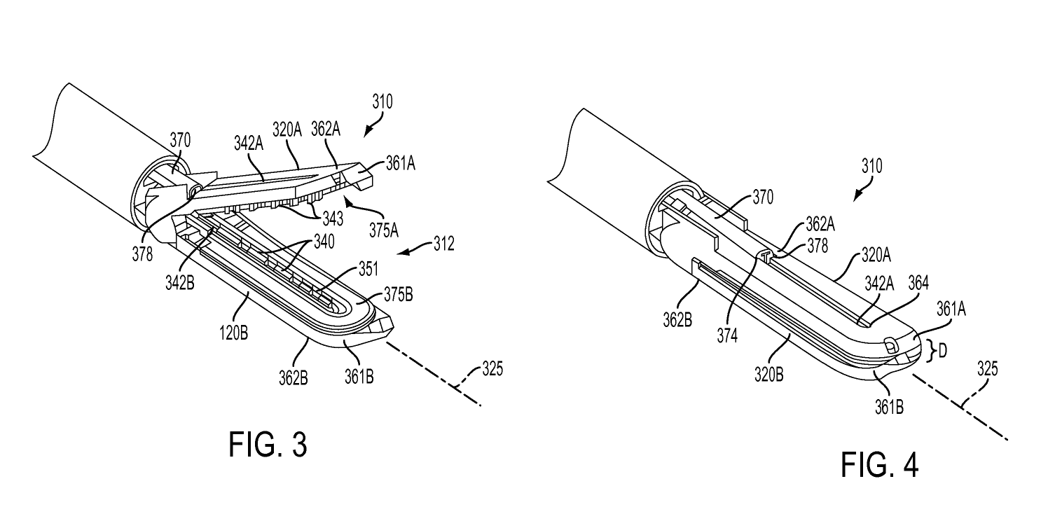

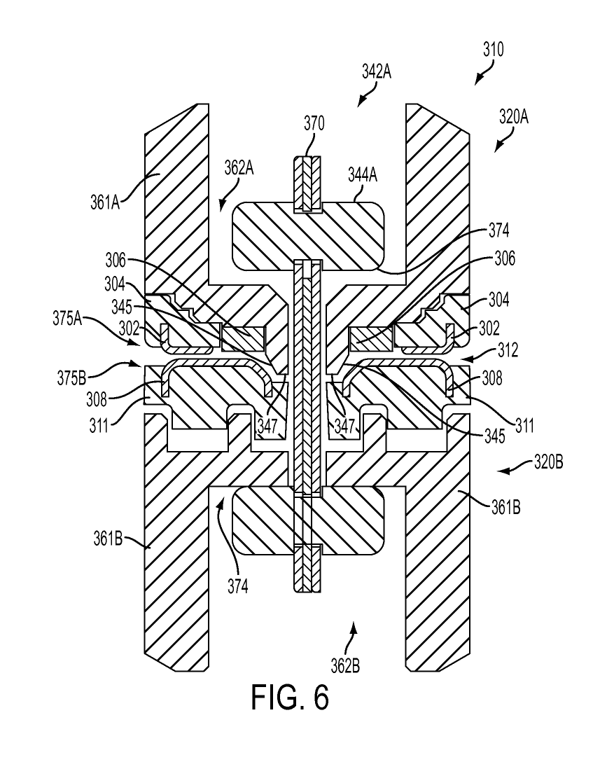

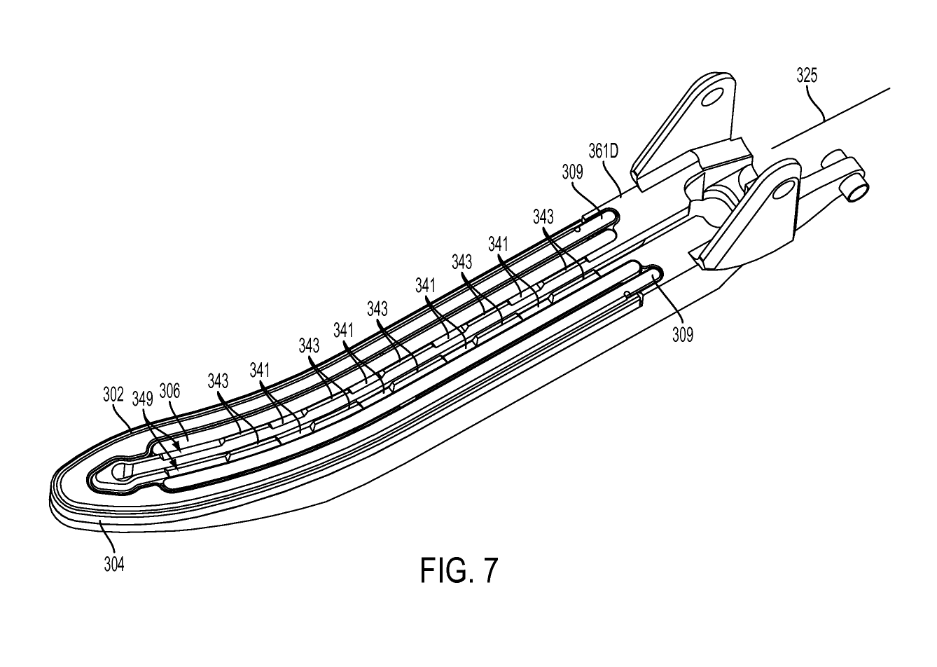

FIGS. 3 and 4 illustrate perspective views of one embodiment of an end effector 310. The end effector 310 may be used with any suitable surgical instrument including, for example, the surgical instrument 102 described herein. FIG. 3 shows the end effector 310 in an open configuration and FIG. 4 shows end effector 310 in a closed configuration. FIG. 5 illustrates perspective cross-sectional view of one embodiment of the end effector 310 in a closed configuration. FIG. 6 illustrates a cross-sectional view of one embodiment of the end effector 310 in the closed configuration. The end effector 310 may comprise the upper first jaw 320A and the lower second jaw 320B. The first jaw 320A and the second jaw 320B each may comprise an elongate slot or channel 342A and 342B, respectively, disposed outwardly along their respective middle portions. The channels 342A, 342B may be parallel to the longitudinal axis 325. The first jaw 320A may comprise an upper first jaw body 361A with an upper first outward-facing surface 362A and an upper first energy delivery surface 375A of a first electrode, for example. The second jaw 320B may comprise a lower second jaw body 361B with a lower second outward-facing surface 362B and a lower second energy delivery surface 375B of a second electrode, for example. The first energy delivery surface 375A and the second energy delivery surface 375B may both extend in a “U” shape about the distal end of end effector 310. The energy delivery surfaces 375A, 375B may provide a tissue contacting surface or surfaces for contacting, gripping, and/or manipulating tissue therebetween.

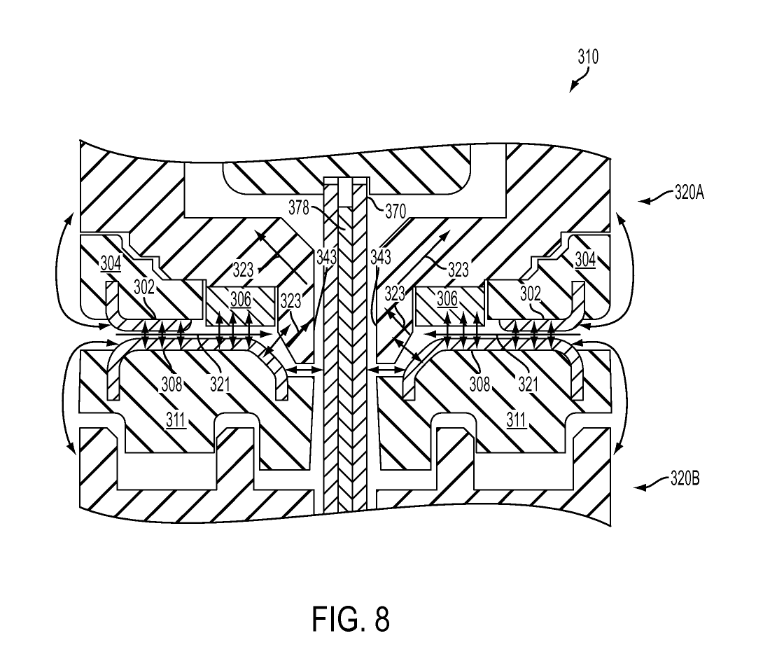

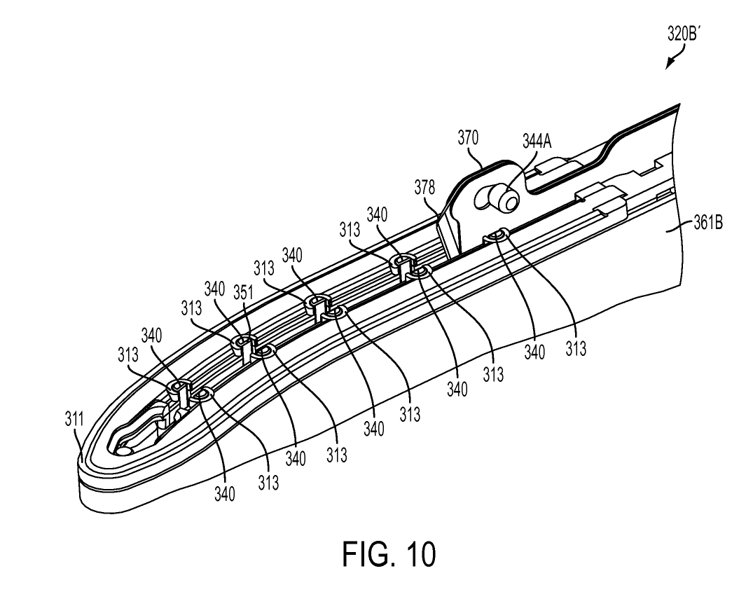

In some examples, the first jaw 320A may have an inner surface 349 adjacent and/or near the channel 342A (FIG. 7). The inner surface 349 may be made of a thermally and/or electrically conductive material, such as a metal. In some examples, the inner surface 349 may be integral with or in direct or indirect contact with the jaw body 361A to conduct heat and/or electricity away from a tissue treatment area 312. In some examples, the inner surface 349 may comprise teeth 343 to grip tissue present between the jaws 320A, 320B. The teeth 343, may comprise multiple surfaces 345, 347, as described herein, and may comprise a thermally conductive material, such as a metal. The teeth 343 may be positioned at regular intervals along a longitudinal axis 325 of the end effector 310. The intervals may be constant or variable. Inter-conductor or inter-tooth recesses 341 may be positioned between the teeth 343 (FIG. 7). In some examples, the second jaw 320B also comprises an inner surface 351 adjacent and/or near the channel 342B (FIG. 3 and FIG. 10). The inner surface 351 may be electrically and/or thermally conductive; may be integral with or in direct or indirect contact with the jaw body 361B and may comprise teeth 340. In some examples (FIG. 7 and FIG. 10), the jaws 320A, 320B may be curved away from the longitudinal axis 325. Features of curved jaws 320A, 320B such as the channels 342A, 342B, electrodes 302, 308, teeth 343, etc., may extend along the length of the jaws 320A, 320B, tracking their curvature.

Referring to FIGS. 3-5, in at least one embodiment, a cutting element 370 may be sized and configured to fit at least partially within the channel 342A of the first jaw 320A and the channel 342B of the second jaw 320B. The cutting element 370 may comprise a distal blade 378 for cutting tissue, as described herein. The cutting element 370 may translate along the channel 342A between a first, retracted position correlating with the first jaw being at the open position (FIG. 3), and a second, advanced position correlating with the second jaw being at the closed position (see, for example, FIG. 4). The trigger 109 of handle 118 see FIGS. 1 and 2, may be adapted to actuate the cutting element 370. The cutting element 370 and/or the distal blade 378 may be made of 17-4 precipitation hardened stainless steel, for example. At least a portion of the cutting element 370 may be 716 stainless steel. The distal portion of the cutting element 370 may comprise a flanged “I”-beam configured to slide within the channels 342A and 342B in jaws 320A and 320B. In various embodiments, the distal portion of the cutting element 370 may comprise a “C”-shaped beam configured to slide within one of channels 342A and 342B. As illustrated in FIGS. 3-5, the cutting element 370 is shown residing in and/or on the channel 342A of the first jaw 320A. The cutting element 370 may slide within the channel 342A, for example, to open and close the first jaw 320A with respect to the second jaw 320B. The distal portion of the cutting element 370 also may define inner cam surfaces 374 for engaging outward facing surfaces 362A of the first jaw 320A, for example. Accordingly, as the cutting element 370 is advanced distally through the channel 342A, from, for example, a first position (FIG. 3) to a second position (FIG. 4), the first jaw 320A may be urged closed (FIG. 4). In some examples, the closure and opening of the first and second jaws 320A, 320B may be performed utilizing a linkage mechanism, as described herein.

The flanges 344A and 344B of the cutting element 370 may define the inner cam surfaces 374 for engaging the outward facing surfaces 362B of the second jaw 320B. As discussed in greater detail herein, the opening and closing of jaws 320A and 320B can apply very high compressive forces on tissue using cam mechanisms which may include reciprocating “C-beam” cutting element 370 and/or “I-beam” cutting member 140 and the outward facing surfaces 362A, 362B of jaws 320A, 320B. In some embodiments, the flanges 344A, 344B may comprise one or more pins extending from and/or through the cutting element 370, for example, as shown in FIG. 6.

More specifically, referring still to FIGS. 3-6, collectively, the flanges 344A and 344B of the distal end of the cutting element 370 may be adapted to slidably engage the second outward-facing surface 362B of the second jaw 320B, respectively. The channel 342A within the first jaw 320A and the channel 342B within the second jaw 320B may be sized and configured to accommodate the movement of cutting element 370. FIG. 4, for example, shows the distal blade 378 of the cutting element 370 advanced at least partially through the channel 342A. The advancement of the cutting element 370 can close the end effector 310 from the open configuration shown in FIG. 3 to the closed configuration shown in FIG. 4. The cutting element 370 may move or translate along the channel 342A between a first, retracted position and a second, fully advanced position. The retracted position can be seen in FIG. 3, where the jaws 320A, 320B are in an open position and the distal blade 378 of the cutting element 370 is positioned proximal to the upper outward-facing surface 362A. The fully advanced position, while not shown, may occur when the distal blade 378 of the cutting element 370 is advanced to a distal end 364 of the channel 342A and the jaws are in a closed position, see FIG. 4.

In at least one embodiment, distal portions of the cutting element 370 may be positioned within and/or adjacent to one or both of the jaws 320A and 320B of the end effector 310 and/or distal to the elongate shaft assembly 112 (see FIGS. 1 and 2). Further, in the closed position shown by FIG. 4, the upper first jaw 320A and the lower second jaw 320B define a gap or dimension D between the first energy delivery surface 375A and the second energy delivery surface 375B of the first jaw 320A and the second jaw 320B, respectively. Dimension D may equal from about 0.0005 inches to about 0.040 inches, for example, and in some embodiments may equal about 0.001 inches to about 0.010 inches, for example. Also, the edges of first energy delivery surface 375A and second energy delivery surface 375B may be rounded to prevent the dissection of tissue.

In various aspects, the upper and lower jaw bodies 361A, 361B may be made from a metal, such as steel, or other heat and/or electricity-conducting material. The jaw bodies 361A, 361B, via inner surfaces 349, 351, may wick heat generated during tissue treatment away from the tissue treatment area 312 between the energy delivery surfaces 375A, 375B. This serves to keep the end effector 310 cool, but also tends to increase the time necessary for tissue sealing. When the end effector 310 is used to seal without cutting, this heat wicking can also reduce the quality of the tissue seal generated by the end effector 310. Accordingly, various embodiments utilize an upper jaw 320A having an electrode 302 on the energy deliver surface 375A that is at least partially isolated thermally from the remainder of the upper jaw body 361A. For example, the upper jaw 320A may comprise a heat barrier 304 positioned between the upper jaw electrode 302 and the upper jaw body 361A. This reduces the surface area of the energy delivery surface 375A that is capable of transmitting heat energy away from the tissue treatment area 312. The upper jaw heat barrier 304 may comprise any suitable material for insulating heat including, for example, plastic, silicon, ceramic, etc. The upper jaw 320A may also comprise a positive temperature coefficient (PTC) 306.

FIG. 7 illustrates the upper jaw 320A showing additional features of the electrode 302 and heat barrier 304. The electrode 302 is illustrated as a single piece wrapping around the jaw 320A. PTC components 306 are shown as two non-contiguous strips. Some embodiments comprise a single PTC component 306 wrapped around the jaw 320A in a “U” shape similar to the electrode 302. The electrode 302 may be manufactured according to any suitable technique or form. In some aspects, the electrode 302 is stamped. In various embodiments, the electrode 302 is secured to the jaw body 361A at connection points 308. For example, the electrode 302 may be welded to the jaw body 361A at connection points 309. The connection points 309 may transmit heat from the electrode 302 to the jaw body 361A. Positioning the connection points 309 away from tissue may prevent the connection points 309 from wicking heat away from tissue. Connection points 309 can be positioned at a part of the jaw 320A that has no contact or minimal contact with tissue to be sealed. As illustrated in FIG. 7, connection points 309 are positioned at proximally positioned portions of the electrode 302 such as, for example, the proximal-most portions of the electrode.

The energy delivery surface 375A of the upper jaw 320A may comprise the inner surface 349, the electrode 302 and the optional PTC component 306. Referring to FIGS. 6-7, the electrode 302 and PTC component 306 may be positioned opposite an electrode 308 of the lower jaw 320B. The electrode 308 may be electrically isolated from the lower jaw body 361B via an insulating layer 311. The insulating layer 311 may prevent direct contact between the electrode 308 and a return path to the RF source (not shown), such as the lower jaw body 361B or any other conductive portions of the lower jaw 320B. The insulating layer 311 may be made from any suitable electrically insulating material including, for example, plastic, silicon, ceramic, etc. In various examples, the insulating layer 311 also insulates the transmission of heat.

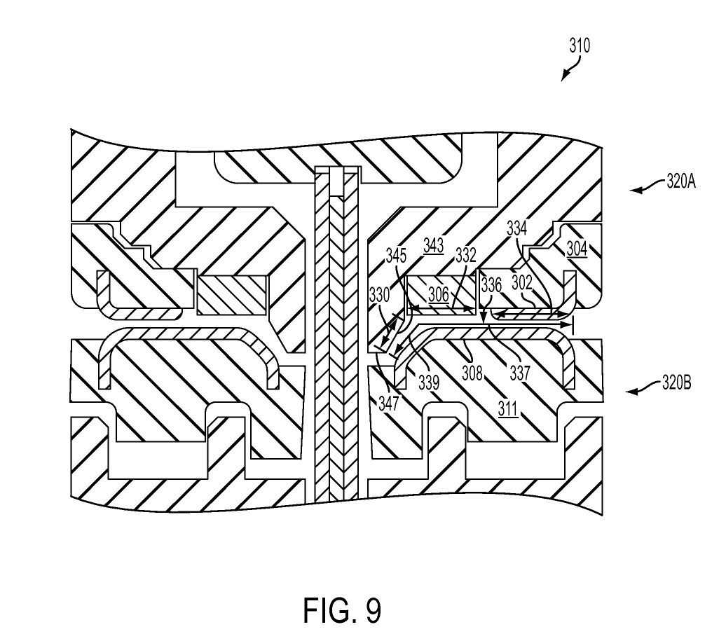

FIG. 8 illustrates a cross-sectional view of one embodiment of the end effector 310 in the closed configuration illustrating current and heat flows in the end effector 310 during tissue treatment. For example, in FIG. 8, the end effector 310 is shown with the jaws 320A, 320B in a closed position. When tissue is treated, it may be placed between the jaws 320A, 320B as indicated by treatment area 312. When the end effector 310 is energized, current may flow from the electrode 308 to a return path to the RF source. The return path may include, for example, the electrode 302, the PTC component 306, the cutting element 370, the jaw bodies 361A, 361B, the teeth 343, etc. The unlabeled arrows in FIG. 8 illustrate potential current paths. For example, current may flow from the electrode 308 through tissue: to the electrode 302; to the PTC component 306; to the upper jaw body 361A around the outside of the end effector 310; to the upper jaw body 361A via a tooth 343 or other interior component; to the lower jaw body 361B via an outside of the end effector 310; to the cutting element 370, etc. In various embodiments, the PTC component 306 may be configured to change its resistance as its temperature increase. For example, when the treated tissue heats the PTC component 306 to a threshold temperature, its heat and electrical conductivity may decline, reducing the energy provided to the tissue.

As current paths pass through tissue in the treatment area 312 between the jaws 320A, 320B, the tissue man be heated. The heat may be wicked away from the treatment area 312 as indicated by arrows 321 and 323. The presence of the insulating layer 304 may prevent heat from dissipating to the jaw body 361A through the electrode 302. Also, the presence of the insulating layer 311 may prevent significant heat from being transmitted via the jaw body 361B. Accordingly, heat from the treatment area 312 may be directed towards the center of the jaws 320A, 320B, for example, towards the inner surface 349 and teeth 343 near the jaw channel 342A and, when present, the inner surface 351 and teeth 340 near the jaw channel 342B, as indicated by heat direction arrow 321. Heat may be wicked by the teeth 343 or other portion of the jaw body 361A in thermal contact with the tissue to the jaw body 361A, as indicated by heat direction arrows 323. Utilizing the insulating layer 304 to direct heat as indicated in FIG. 8 may reduce, but not eliminate, the flow of heat away from the tissue through the jaw body 361A. For example, the rate of heat flow may be fast enough to prevent the tissue from becoming too hot too fast and sticking to the electrodes 302, 308 but slow enough to allow acceptable treatment times. In some examples, as illustrated, the inner surface 349 and teeth 343 may be positioned near the channel 342A where the cutting element 370 and knife 378 pass. This may facilitate the removal of heat from the channel 342A near the knife 378. The resulting reduction in heat near the channel 342A and knife 378 may reduce the risk of tissue sticking to the knife 378 or channel 342A.

The rate of heat flow from the tissue treatment area 312 may be based on the relative dimensions of the components of the end effector 310. For example, the lower electrode 308 may be electrically and thermally isolated from the remainder of the lower jaw 320B by the insulator 311. Accordingly, heat flow away from the treatment area 312 may be via the heat conducting components of the upper jaw 320A such as the inner surface 349 and, before its conductivity drops, the PTC component 306. The rate of heat flow from the treatment area 312, then, may depend on the percentage or portion of the lower electrode 308 that is opposite a heat insulating component (e.g, the electrode 302), the percentage or portion of the lower electrode 308 that is opposite a partial conductor of heat (e.g., the PTC component 306) and the percentage or portion of the lower electrode 308 that is opposite a heat conductor (e.g., the surface 345 of the tooth 343).

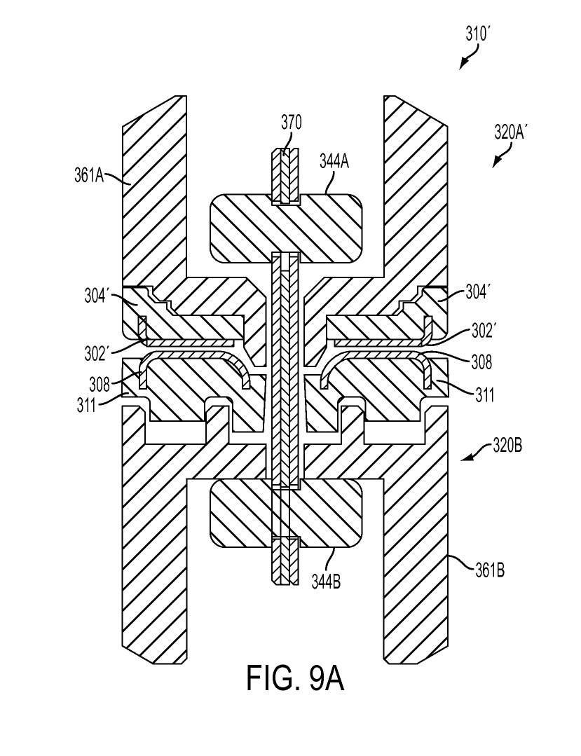

FIG. 9 illustrates a cross-sectional view of one embodiment of the end effector 310 in the closed configuration showing example dimensions for various components of the end effector. The heat transfer capability of the end effector 310 may be described, then by a ratio of the surface area of the upper jaw components capable of transmitting and positioned to transmit heat to the surface area of the lower jaw electrode 308. The upper jaw components capable of transmitting heat, as described above, may include the teeth 343 and the PTC component 306 before its electrical and heat conductivity drops during treatment. The surfaces of these components that are best positioned to transmit heat may be the surfaces that are opposite the electrode 308. For example, the tooth 343 may have multiple surfaces. A first surface 345 is positioned opposite the electrode 308 while a second surface 347 is oriented towards the insulator 311. Accordingly, the surface area of the first surface 345 may be considered to determine the heat transfer properties of the teeth 343. Also, when the teeth 343 are not continuous along the inner surface 349, the heat transmitting effect of the teeth may be multiplied by a fraction indicating the duty cycle of the teeth, or portion of the length of the jaw 320A that comprises teeth 343 instead of inter-teeth recesses 341.

In the examples shown herein, the electrode 308, electrode 302, PTC component 306, and teeth are shaped such that ratios of the surface areas of the various components may be approximated by ratios of the lengths of components surfaces in a direction perpendicular to the longitudinal axis. The lower electrode 308 may have two surfaces, a first surface 337 is shown in FIG. 9 to be about parallel to the electrode 302 and PTC component 306. A second surface 339 is shown to be about parallel to the surface 345 of the tooth 343. A width 336 of the lower electrode 308 as shown in FIG. 9 is the sum of the width of the first surface 337 and the second surface 339. Accordingly, the width 336 of the lower electrode 308 may describe the width of the surfaces 337, 339 of the lower electrode 308 that are in contact with tissue during treatment. The width 336 may equal from about 0.060 inches to about 0.090 inches, for example, and in some embodiments, the width 336 may equal about 0.077 inches.

The upper electrode 302 may be positioned opposite at least a portion of the width 336 of the lower electrode 308. As shown in FIG. 9, the upper electrode 302 has a single surface on the energy delivery surface 375A that is opposite and parallel to the surface 337 of the lower electrode 308. In some examples, the width 334 of the electrode 302 may be between about 39% and 45% of the width of the lower electrode 308, for example about 42%. In some examples, the width 334 of the electrode 302 may be between about 0.030 and 0.035 inches, for example about 0.033 inches. The PTC component 306 may also have a surface on the energy delivery surface 375A that is opposite and parallel to the surface 337 of the lower electrode 308. In some examples, the width 332 of the PTC component 306 may be between about 33% and 39% of the width 336 of the lower electrode 308, for example, about 36%. The width 332 of the PTC component 306 may be between about 0.025 and 0.030 inches, for example, about 0.028 inches. In some examples, the surface 345 of the tooth 343 opposite the surface 339 of the electrode 308, indicated by 330, may have a width between about 19% and 25% of the width 336 of the electrode 308, for example, 22%. The surface 345 may have a width of between about 0.015 and 0.020 inches, for example, about 0.016 inches.

FIG. 9A illustrates a cross-sectional view of one embodiment of the end effector 310′ in the closed configuration showing an alternate upper electrode 302′. For example, the upper jaw 120′ of the end effector 310′ may omit the PTC component 306. Instead, the end effector 310′ may comprise the extended upper electrode 302′ extending to the inner surface 349 (shown as tooth 343 in FIG. 9A). In some examples, the sum of the width of the extended upper electrode 302′ and the surface 345 of the tooth 343 may be about equal to a width of the electrode 308. An extended insulator 304′ may be positioned, for example as shown, to electrically and thermally insulate the electrode 302′ from the remainder of the jaw 320A. In the end effector 310′, heat transfer away from the treatment area 312 may be primarily through the teeth 343. Accordingly, the rate of heat transfer may be based on the dimensions of the teeth 343 such as, for example, the ratio of the width of the surface 345 opposite the electrode 308 to the width of the electrode 308.

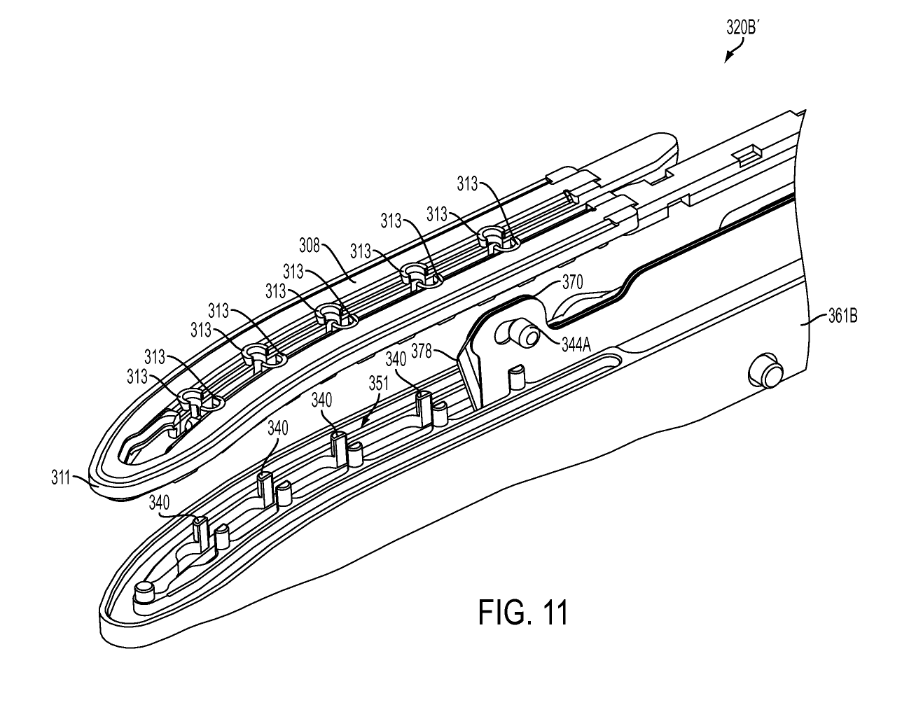

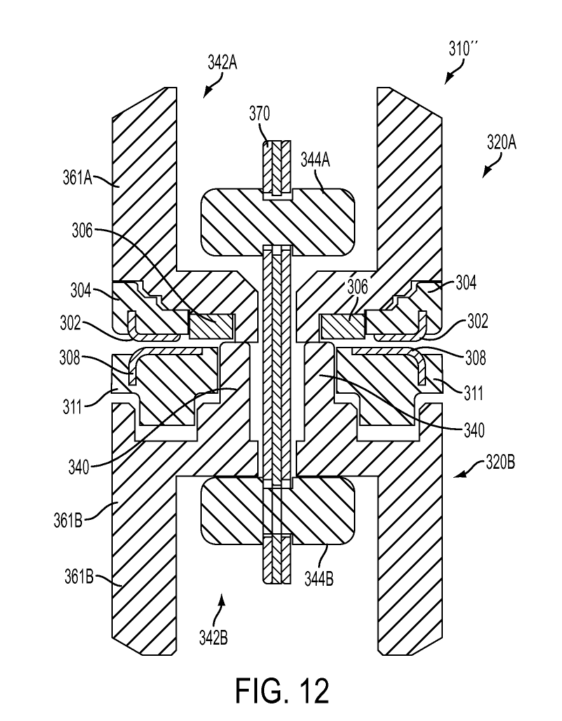

In some examples, the lower jaw of the end effector may also provide a path for heat transfer away from the tissue treatment area 312 via the inner surface 351. The path provided by the lower jaw may be in addition or alternative to the paths via the teeth 343 and PTC component 306 described herein. FIG. 10 illustrates one embodiment of the lower jaw 320B′ comprising an inner surface 351 with conductive teeth 340. FIG. 11 illustrates an exploded view of one embodiment of the lower jaw 320B′. FIG. 12 illustrates a cross-sectional view of one embodiment of an end effector 310″ comprising the lower jaw 320B′ taken at a set of the conductive teeth 340. The teeth 340 may be thermally conductive to direct heat away from the tissue treatment area 312. As shown, the teeth 340 may be positioned at any suitable interval along the length of the jaw 320B′. In some examples, including the one shown in FIGS. 10-12, the teeth 340 may be arranged in pairs, with each pair straddling the channel 342B. Placing the teeth 340 near the channel 342B and knife 378 may facilitate increased heat flow from the channel 342B and knife 378 which can minimize tissue sticking to the channel 342B and knife 378 during treatment.

In some examples, the inner surface 351, including the conductive teeth 340, is a part of and/or a contiguous component of the lower jaw body 361B (FIG. 11). To prevent shorting of the electrode 308 to the jaw body 361B, the conductive teeth may be insulated from the lower jaw body 361B by insulating layer 313 (FIG. 10). The insulating layer 313 may comprise discrete insulators or, in some examples, may include an extended portion of the insulator 311 (FIGS. 11 and 12). Referring now to FIG. 12, the conductive teeth 340 may be positioned opposite a corresponding portion of the upper jaw 320A. In some examples, the lower jaw 320B′ may be utilized with an upper jaw having flat teeth C42 positioned to rest on the teeth 340 when the end effector 110″ is closed as shown in FIG. 12. In other examples, the teeth 340 may be positioned to offset the teeth 343 such that the teeth 340 rest in inter-tooth recesses 341 (FIG. 7) between the teeth 343.

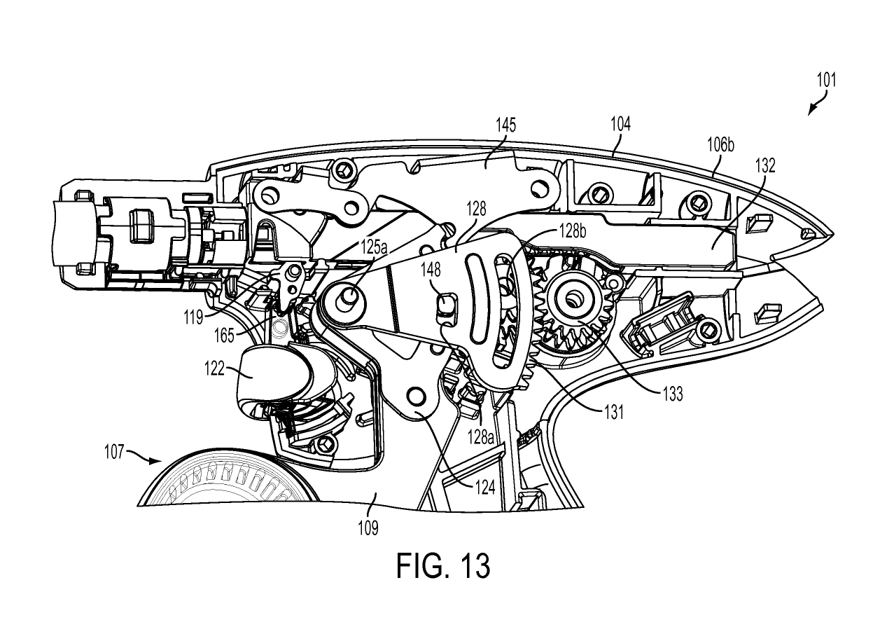

The description now turns to various example embodiments of surgical instruments in which the electrosurgical devices with electrode mechanisms configured for heat management can be practice. Accordingly, turning now to FIG. 13 is a side elevation view of a handle assembly 104 of a surgical instrument 101, with the left handle housing shroud 106a removed to expose various mechanisms located within the handle assembly 104 and without the knife lockout disabling mechanism 108, according to one embodiment. Except for the knife lockout disabling mechanism, in other aspects, the surgical instrument 101 operates in a manner similar to the surgical instrument described in connection with FIGS. 1 and 2.

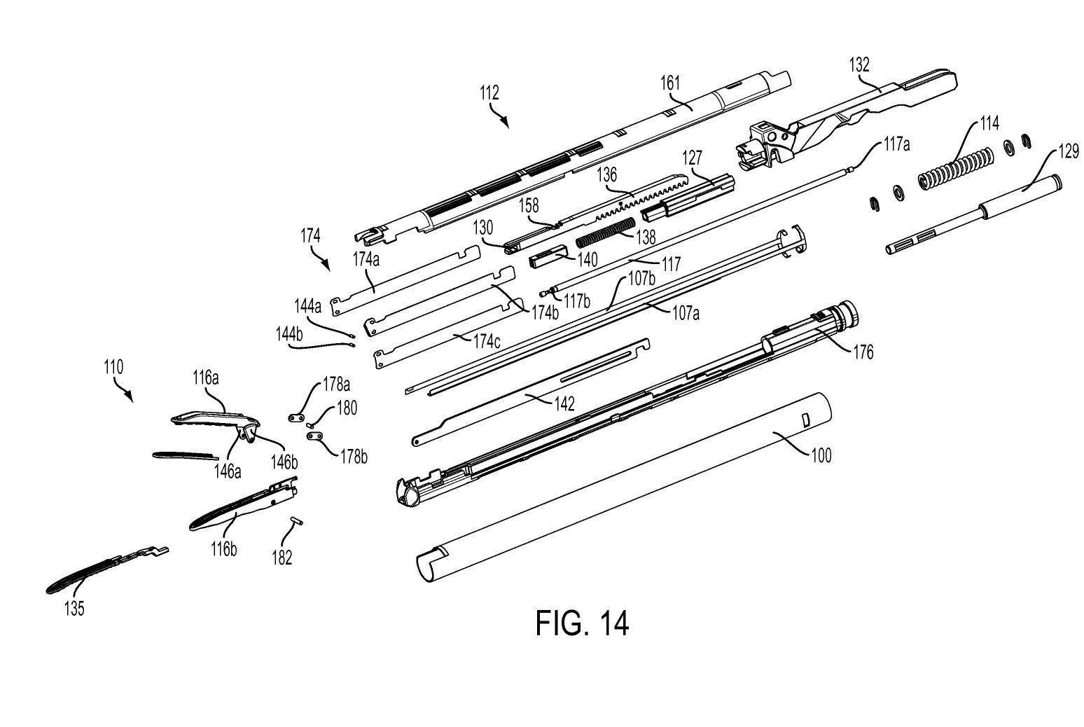

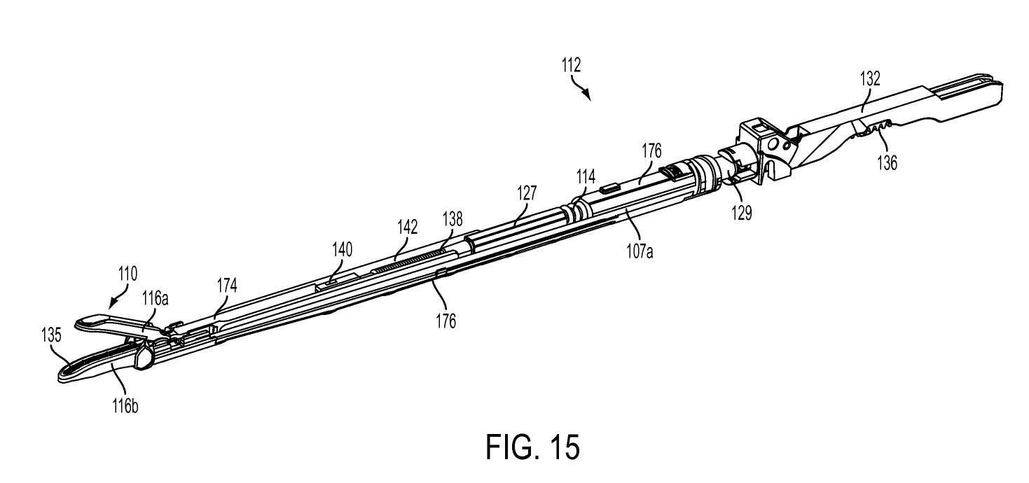

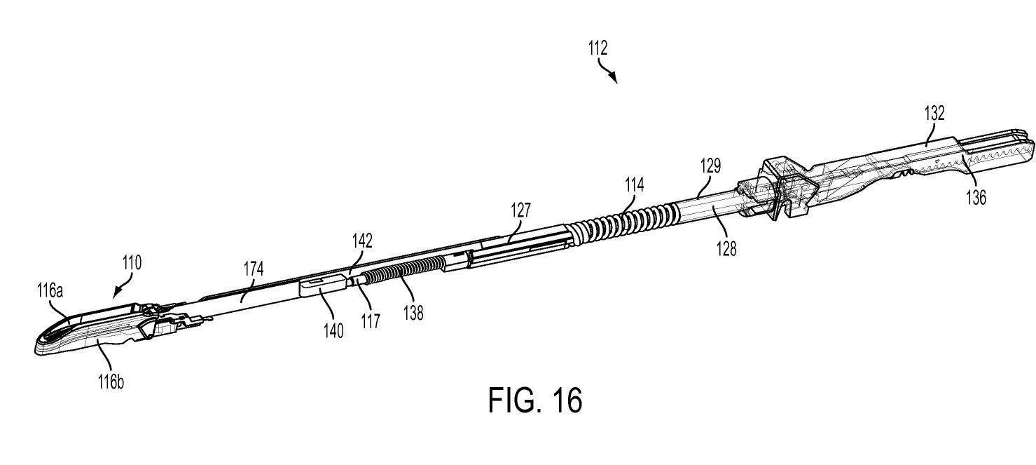

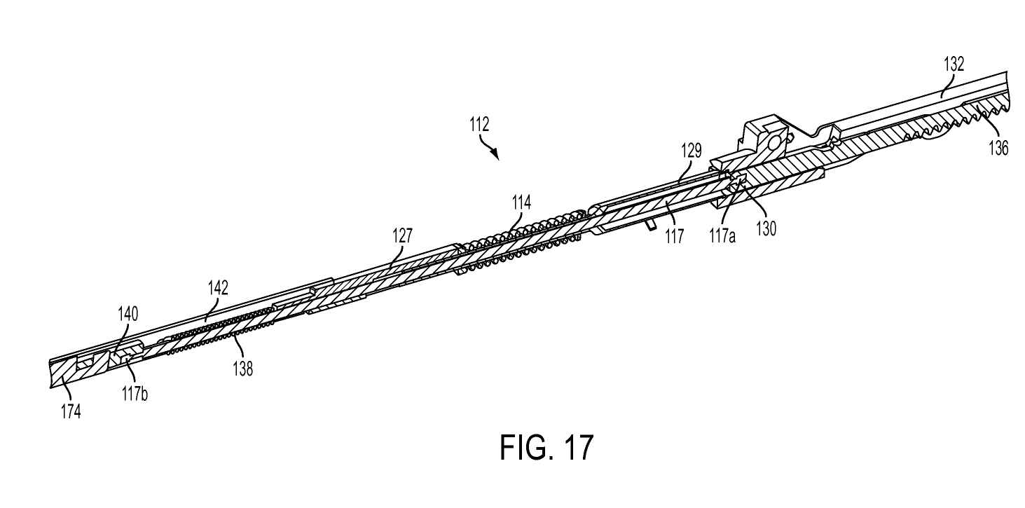

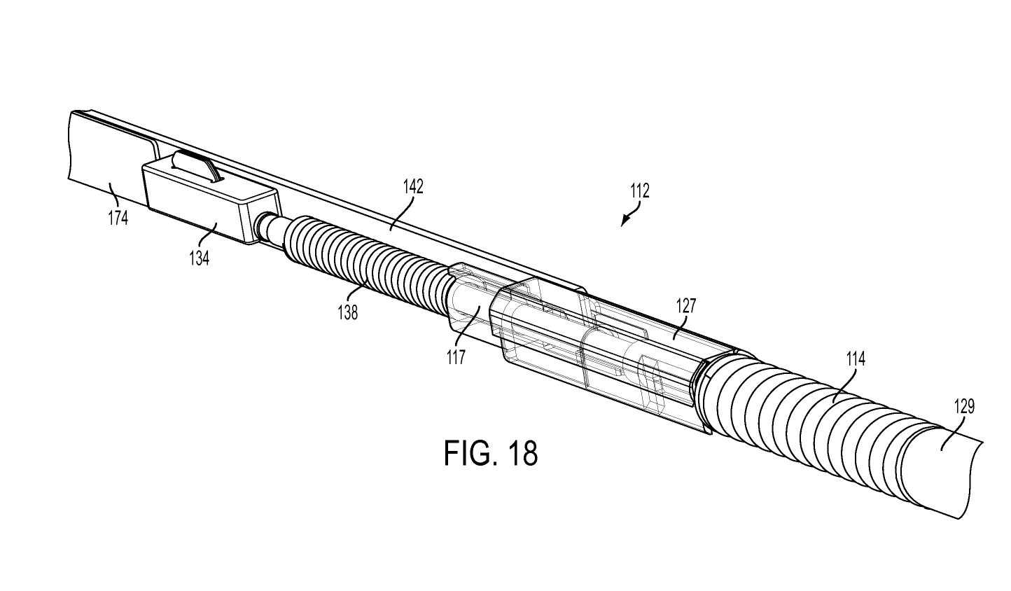

FIG. 14 is an exploded view of the shaft assembly 112, end effector 110, yoke 132, and rack 136 portions of the surgical instrument 102 shown in FIGS. 1 and 2, according to one embodiment. FIG. 15 is a perspective view of the shaft assembly 112, end effector 110, yoke 132, and rack 136 shown in FIG. 4 in the assembled state, according to one embodiment. FIG. 16 is a perspective view of the shaft assembly 112, end effector 110, yoke 132, and rack 136 shown in FIG. 15, according to one embodiment, with the electrically insulative nonconductive tube 176 removed to show the functional components of the shaft assembly 112 in the assembled state. FIG. 17 is a sectional view taken along a longitudinal axis of the shaft assembly 112, yoke 132, and rack 136 shown in FIG. 15, according to one embodiment, to show the functional components of the shaft assembly 112 in the assembled state. FIG. 18 is partial perspective view of the shaft assembly 112 shown in FIG. 17, according to one embodiment.

With reference now to FIGS. 14-17, the shaft assembly 112 comprises an outer tube 100 which contains or houses the various functional components of the shaft assembly 112. An electrically insulative nonconductive tube 176 is slidably received within the outer tube 100. A clamp tube 161 is attached to the nonconductive tube 176. The functional components of the shaft assembly 112 are slidably contained within the within the nonconductive tube 176 whereas the conductive elements 107a, 107b employed to supply electrical energy to the end effector 110 electrodes 135 are located outside the nonconductive tube 176. A closure actuator 129 is coupled to the distal end of the yoke 132. The closure actuator 129 comprises a proximal portion and a distal portion. The distal portion of the closure actuator 129 is sized to be received within a closure spring 114. The proximal portion of the closure actuator 129 is sized to compress the closure spring 114. The closure spring 114 is coupled to a closure bar 142 through a spring to bar interface element 127. The distal end 172 of the closure bar 142 is operatively coupled to the jaws 116a, 116b by a pin 180 and closure linkages 178a, 178b. The jaws 116a, 116b are pivotally coupled by a pin 182 and rotatable support structures 146a, 146b formed in the top jaw 116a. The closure actuator 129 is coupled to the distal end of the yoke 132, which is operatively coupled to the toggle clamp 145 (FIGS. 1, 2, and 13, for example). As previously described, the toggle clamp 145 is movably coupled to the trigger plate 124 (FIGS. 1, 2, and 13), for example. Rotation of the trigger plate 124 straightens the toggle clamp 145 to drive the yoke 132 distally. Distal movement of the yoke 132 causes distal movement of the closure actuator 129 to compresses the closure spring 114 and drive the closure bar 142. Distal movement of the closure actuator 142 pivotally moves the first jaw member 116a from an open position to a closed position with respect to the second jaw member 116b, for example.

A firing bar 117 comprises a proximal end 117a and a distal end 117b. The proximal end 117a of the firing bar 117 is coupled to the distal end 130 of the rack 136. The rack 136 is received within the yoke 132. The firing bar 117 is received within the closure actuator 129, the spring to bar interface element 127, and the jaw open spring 138. The distal end 117b of the firing bar 117 is fixedly coupled to a knife pusher block 140, which is fixedly coupled to a cutting element 174 (knife). The cutting element 174 comprises flexible bands 174a, 174b, 174c, which are fastened by the knife pusher block 140 at the proximal end and by pins 144a, 144b at the distal end to form knife or cutting element having an I-beam configuration. As previously described, the teeth 131 of the sector gear of the firing plate 128 engage and rotate the pinions 133, 134, which drive the rack 136 distally. The rack 136 drives the firing bar 117, which in turn drives the flexible I-beam cutting element 174 when the lock arm 157 is disengaged from a notch 158 formed in the rack 136.

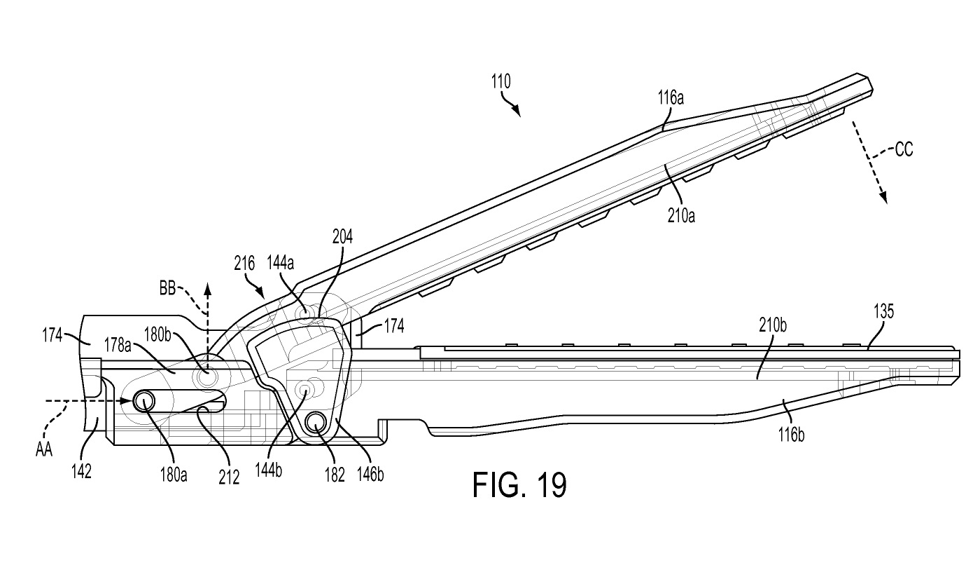

FIG. 19 is a side view of an end effector 110 portion of the surgical instrument 102 shown in FIGS. 1 and 2 with the jaws open, according to one embodiment. The closure bar 142 is operatively coupled to the proximal end of the top jaw 116a via the closure linkages 178a, 178b (not shown) and first and second pins 180a, 180b. The lower pin 180a is slidably movable within a slot 212. As the closure bar 142 moves distally in the direction indicated by arrow AA, the pin 180a slides in the slot 212 to and forces the second pin 180b to move upwardly in the direction indicated by arrow BB to force the top jaw 116a to rotate to a closed position as indicated by arrow CC. The top jaw 116a pivots about a pivot point defined by the fastener pin 182. The bottom jaw 116b comprises the electrode 135, which is electrically coupled to an energy source (e.g., an RF electrosurgical energy source). The flexible I-beam band knife comprises a knife or cutting element 174. The cutting element 174 and the fastener pins 144a, 144b form an I-beam member 216 that forces the jaws 116a, 116b shut when the cutting element 174 is fired by the rack 136 and firing bar 117, as previously described. The I-beam member 216 advances distally on tracks 210a, 210b formed in the respective upper and lower jaws 116a, 116b to force the jaws 116a, 116b shut and compress the tissue located therebetween. A ramp 204 is defined at the proximal end of the top track 210a in the top jaw 116a. Accordingly, a predetermined force is required to advance the I-beam member 216 over the ramp 204 before the I-beam member 216 engages the top track 210a to close the jaws 116a, 16b as the I-beam member 206 is advanced distally by the flexible I-beam band 142. In the present view, the I-beam member 216 is located behind the ramp 204 as the linkages 178a, 178b (not shown) close the jaws 116a, 116b.

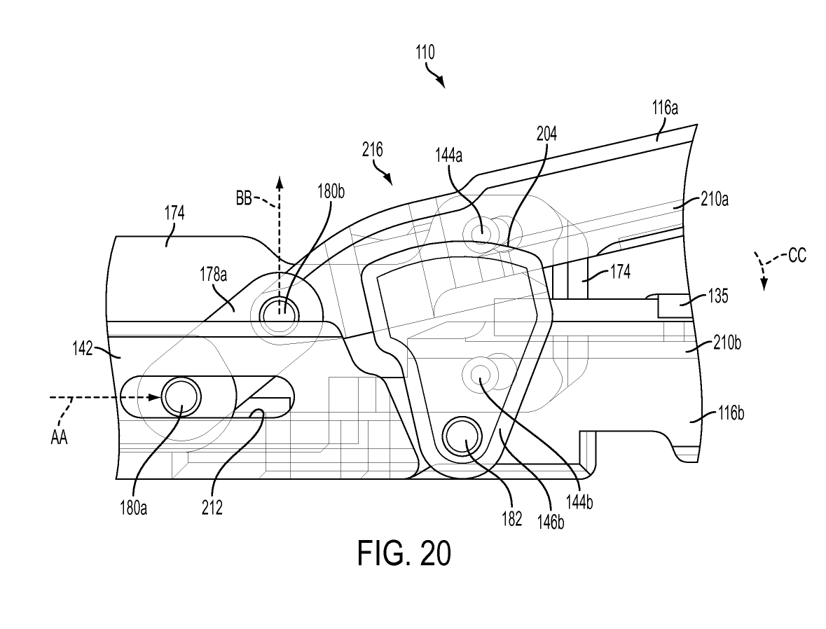

FIGS. 20-23 illustrate a sequence of firing the I-beam member 216 and closure spring 114 driven cam system to simultaneously close a set of opposing jaws 116a, 116b. FIG. 20 shows the closure bar 142 and the I-beam member 216 at the initial stage of clamp closure and firing sequence where the I-beam member 216 is located behind or at the base of a ramp 204 in the upper jaw 116a, according to one embodiment. The pins 144a, 144b (not shown) of the I-beam member 216 are located at the base of the ramp 204 prior to firing the cutting element 174. In this view, the I-beam member 216 is located behind the ramp 204 as pivoting link 178a closes the upper jaw 116a in direction CC.

FIG. 21 shows the closure bar 142 and I-beam member 216 further advanced distally in direction AA than shown in FIG. 20, where the I-beam member 216 is located at an intermediate position along the ramp 204 in the upper jaw 116a, according to one embodiment. FIG. 21 shows the closure bar 142 pushing on the bottom pin 180a to move distally in direction AA within the slot 212. In response, the pivoting link 178a moves distally in direction AA and rotates counterclockwise pushing the top pin 180b upwardly in direction BB to apply a closing force to the upper jaw 116a. The I-beam member 216 also advances partially up the ramp 204. The upper jaw 116a rotates slightly in direction CC toward a closed position.

FIG. 22 shows the closure bar 142 and the I-beam member 216 further advanced distally in direction AA than shown in FIG. 21 where the I-beam member 216 is located at the top of the ramp 204 in the upper jaw 116a, according to one embodiment. In FIG. 22, the closure bar 142 is advanced further distally in direction AA in response to the closure actuator 129 acting on the closure spring 114 and continues pushing on the bottom pin 180a causing it to move further distally in direction AA within the slot 212. In response, the pivoting link 178a moves distally in direction AA and continues rotating counterclockwise pushing the top pin 180b upwardly in direction BB to apply a closing force to the upper jaw 116a. The upper jaw 116a continues rotating further in direction CC toward a closed position. At this stage, the I-beam member 216 is located at the top of the ramp 204.

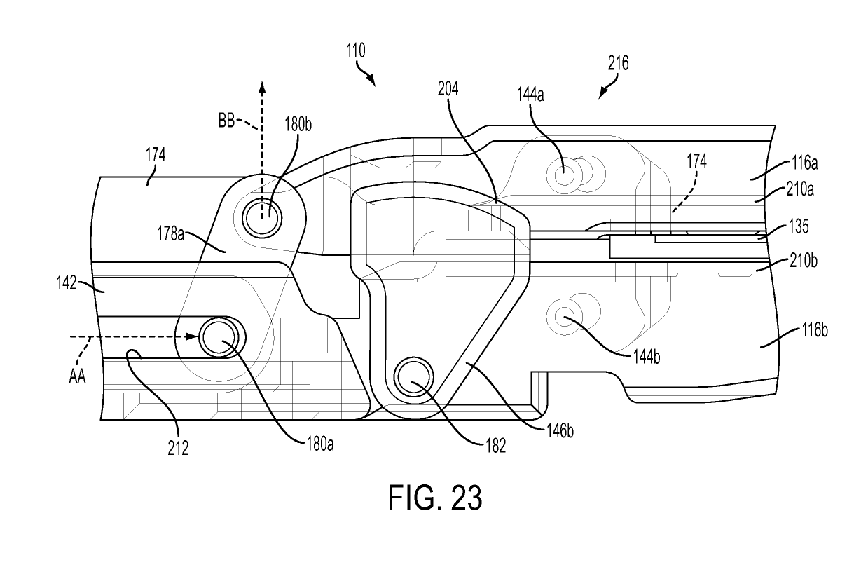

FIG. 23 shows the closure bar 142 and I-beam member 216 further advanced distally than shown in FIG. 22, where the I-beam member 216 is located past the ramp 204 in the upper jaw 116a, according to one embodiment. FIG. 23 shows the closure bar 142 advanced still further distally in direction AA and continues to push on the bottom pin 180a causing it to move distally in direction AA within the slot 212. In response, the pivoting link 178a moves distally in direction AA and continues rotating counterclockwise pushing the top pin 180b upwardly in direction BB to apply a closing force to the upper jaw 116a. The upper jaw 116a continues rotating further in direction CC toward a closed position. In FIG. 23, the I-beam member 216 is located past the ramp 204 and the upper jaw 116a is fully closed in response to the trigger plate 124 acting on the toggle clamp 145, which acts on the yoke 132, and advances the closure actuator 129 and the closure bar 142 to push on the pivoting link 178a. The I-beam member 216 pins 144a, 144b are now located past the ramp 204 and are located in the tracks 210a, 210b formed in the respective upper and lower jaws 116a, 116b. The I-beam member 216 is now prepared to slide distally in direction AA. In response to the trigger 109 being squeezed, the firing plate 128 rotates to advance the rack 136 distally, which acts on the firing bar 117 and pushes the I-beam member 216 and the cutting element 174 distally in direction AA. This action forces the jaws 116a, 116b fully shut to compress the tissue located therebetween.

With reference now to FIGS. 1, 2, and 23, the disclosure now turns to a description of the electrosurgical instrument 102 having a separate spring driven cam closure mechanism for closing the jaws 110 that is independent of the I-beam 216 closure mechanism. In various embodiments, the present disclosure provides an electrosurgical radio frequency (RF) bipolar sealing device comprising a separate spring driven cam closure mechanism that is independent of the I-beam member 216 closure mechanism to simultaneously close a set of opposing jaws 116a, 116b. The spring driven cam closure system can close the jaws 116a, 116b first unless the force to close the jaws 116a, 116b overcomes a spring force. At this point, the I-beam member 216 closure system will close the jaws 116a, 116b. The spring driven cam closure mechanism comprises a spring 114 connected to a bar 127, which is in turn connected to a pivoting link 178a, which is then connected to a jaw 116a. Pushing on the spring 114 pushes on the bar 127, which pushes on the pivoting link 178a which closes the jaw 116a. The spring 114 of the cam closure system can be pre-compressed to raise its starting load.

The closure system of the electrosurgical instrument 102 comprises a first closure system comprising a spring driven cam closure mechanism and a second closure system comprising an I-beam closure mechanism. Both the first and second closure mechanisms are operated by the single trigger 109. The first closure system, otherwise referred to herein as a cam closure system, is driven by the closure of the trigger 109 in the first ˜13 degrees of stroke. During the first stroke of the trigger 109, the trigger plate 124 drives the toggle clamp 145 and yoke 132 to advance the closure actuator 129 distally to compress the closure spring 114. The closure spring 114 drives the closure bar 142 which drives the pin 180a and the pivoting link 178a distally to close the upper jaw 116a independently of the I-beam closure mechanism. It should be noted that during the first stroke of the trigger 109, the rack 132 moves slightly distally to allow the driving bar 117 to push the I-beam member 216 from the base of the ramp 2014 to the top of the ramp 204. The second closure system is driven by the closure of the trigger in the last ˜29 degrees of stroke. During the second stroke of the trigger 109, when the knife lockout mechanism is either unlocked or disabled, the firing plate 128 drives the first and second pinions 133, 134, which drives the rack 136 distally. The rack 136 is fixedly coupled to the firing bar 117, which drives the I-beam member 216 comprising the flexible cutting element 174.

The first closure system is configured to close the set of opposing jaws 116a, 116b in the end effector 110 using the closure spring 114 to drive the closure bar 142 to drive the pin 180a and the pivoting link 178a distally and close the upper jaw 116a onto the lower jaw 116b. The first closure system can apply more clamping force to the jaws 116a, 116b independently of the second closure system that employs the I-beam member 216 to close the jaws 116a, 116b. The additional closing force that is applied by the first closure system provides better grasping force between the jaws 116a, 116b than simply relying on the I-beam member 216 providing the initial closure force to the jaws 116a, 116b by moving the I-beam member 216 up to the top of the ramp 204.

To ensure that the I-beam member 206 will also be able to close the jaws 116a, 116b, the first and second closure systems operate in tandem. Thus, the cam closure drive system comprising the closure bar 142 and pivoting link 178a and I-beam drive system comprising the I-beam member 216 and firing bar 117 operate in tandem. In one embodiment, the cam closure system closes the jaws 116a, 116b and moves along with the I-beam member 216. Some conventional electrosurgical devices employ the toggle clamp 145 to move the I-beam member 216 to close the jaws 116a, 116b. In the present embodiment, however, the first closure system is operably coupled to the toggle clamp 145 in conjunction with the second closure system such that both closure systems advance distally at the same time. The cam closure system can be timed to close the jaws 116a, 116b before the I-beam member 216. The cam closure system also can incorporate an inline closure spring 114. The inline closure spring 114 can compress at the end of the closure stroke (after the first ˜13 degrees of stroke of the trigger 109) to keep the jaws 116a, 116b shut with a set spring force.

When material is located between the jaws 116a, 116b, the closure spring 114 will be compressed over a predetermined limit during the first stroke closure phase of the cam closure system. After the predetermined limit, the I-beam member 216 closure system takes over the function of closing the jaws 116a, 116b. Accordingly, in the illustrated system the I-beam member 216 will ensure that the jaws 116a, 116b always fully close using jus the toggle clamp 145. The I-beam member 216 is configured to only close the jaws 116a, 116b when the material located between the jaws 116a, 116b takes more force to close than the cam closure spring 114 can provide. The cam system also provides a rising mechanical advantage as the upper jaw 116a is closed such that the more compression force is applied to the closure spring 114 the less force is exerted on the jaws 116a, 116b to prevent damaging tissue from too much spring force.

In one embodiment, the closure system comprising a first closure system (spring cam closure system) and a second an I-beam and spring driven cam system to simultaneously close a set of opposing jaws can be configured to operate in the following manner: (1) place tissue in the jaws and pull the trigger; (2) the toggle clamp pushes on the I-beam and the cam closure; (3) the cam immediately pushes on the jaw through a spring to close it, the I-beam trails a closure ramp on the upper jaw; (4) the cam fully closes the jaws before the toggle stops moving; (5) the toggle clamp continues to move (e.g., another 0.05 inches) to compress the closure spring to ensure the jaws are sprung closed, the I-beam moves over the top of the ramp; and (6) thick tissue in the jaws may compress the spring on the cam closure before the end of the toggle stroke, the I-beam will hit the closure ramp and force the jaws closed to ensure that the I-beam will repeatedly be located over the ramp with the jaws closed before the toggle stops moving.

The disclosed closure system comprising a first spring driven cam closure system and a second I-beam driven closure system is configured to simultaneously close a set of opposing jaws 116a, 116b and provides several advantages over conventional devices. The disclosed device is capable of sealing tissue without necessarily cutting the tissue, provides improved tissue grasping without actuating the cutting element 174, locates the I-beam member 216 over the ramp 204 before the I-beam 216 gear train takes over to provide lower force to fire the cutting element 174. The disclosed closure system also provides improved jaw 116a opening and tissue dissection over conventional devices. The disclosed closure system also provides lower force to fire from preload on tissue. The gears coupled to the firing plate 128 fire the I-beam member 216 distally and can be configured to operate with conventional electrosurgical jaw designs. Additional advantages, not necessarily described herein, are also provided.

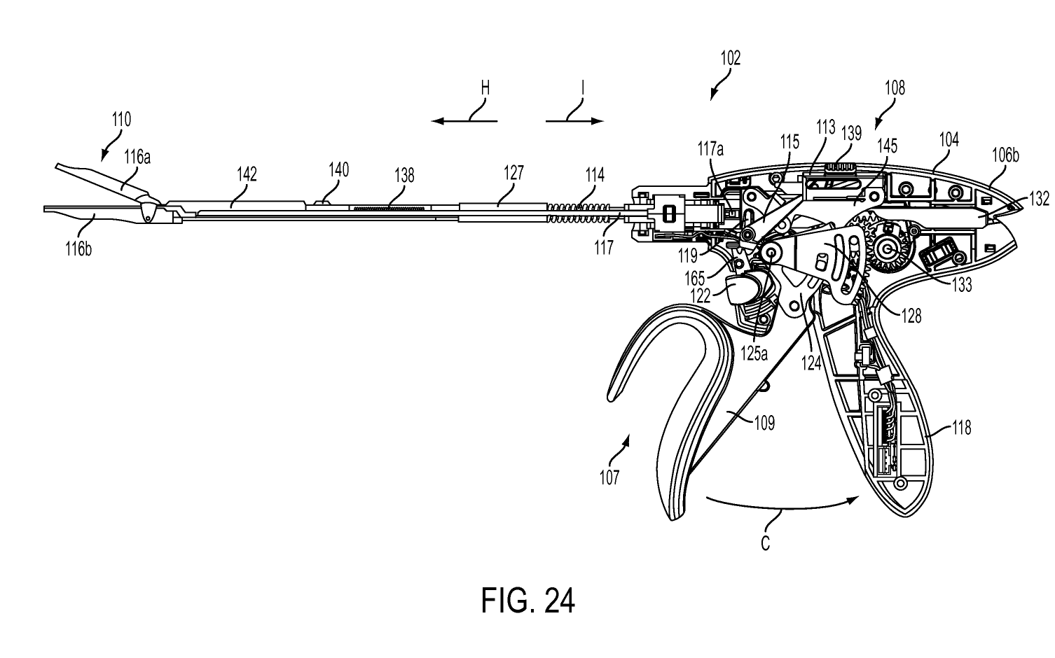

FIGS. 24-33 provide a general description of the surgical instrument 102 shown in FIGS. 1 and 2 comprising a first spring driven cam closure system that operates independently of a second I-beam driven closure system. FIGS. 24-33 illustrate the surgical instrument 102 shown in FIGS. 1 and 2 with the jaw 110 fully open and the lockout defeat mechanism 108 enabled, e.g., in the “ON” position. FIG. 24 is a side elevational view of the surgical instrument 102 shown in FIGS. 1 and 2 with the left housing 106a shroud removed, shaft assembly 112 sheaths removed, the jaw 110 fully open and the lockout defeat mechanism 108 enabled, e.g., in the “ON” position, according to one embodiment. Thus, the button 139 portion of the slider 113 is slidably moved proximally to locate it in the B position.

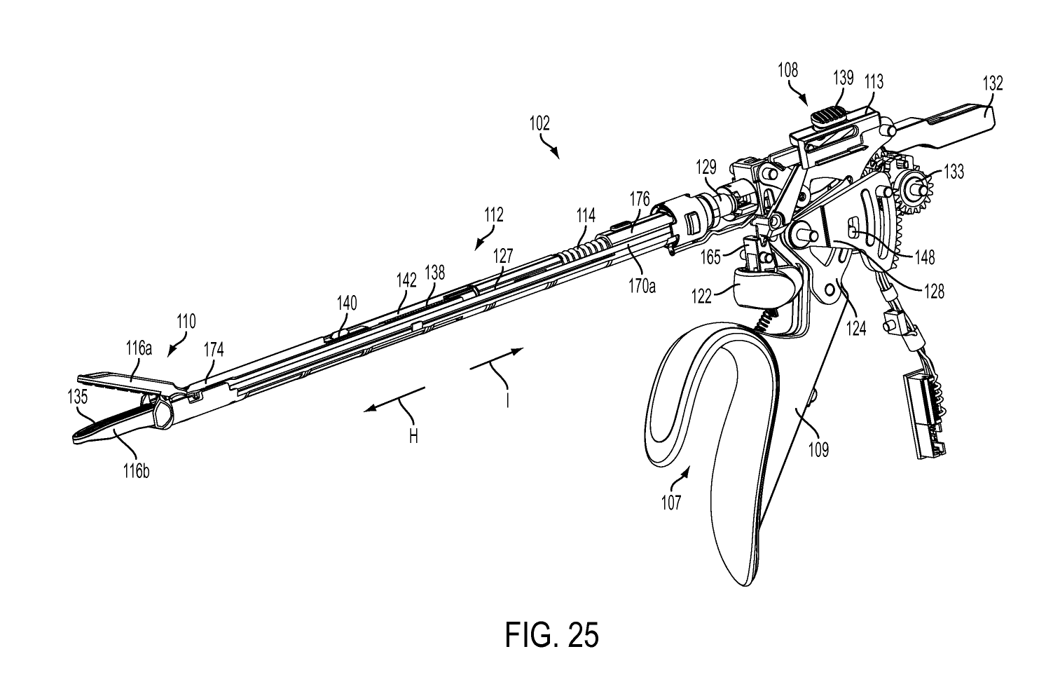

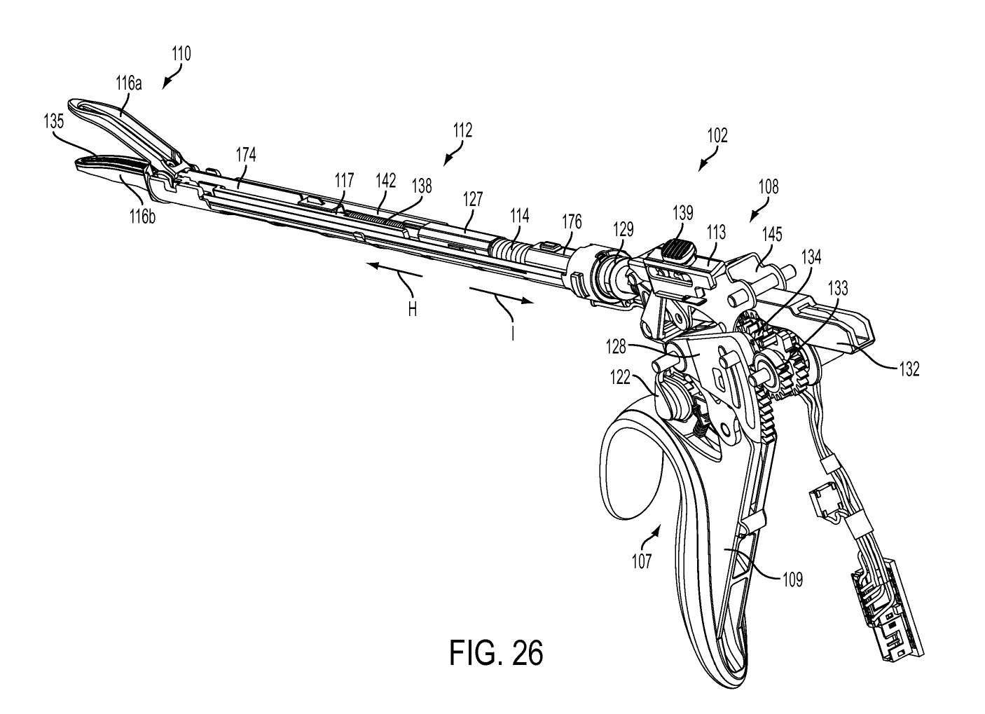

FIG. 25 is a perspective view of the surgical instrument 102 shown in FIG. 24 with the right housing shroud 106b removed, according to one embodiment. FIG. 26 is a perspective view of the surgical instrument 102 shown in FIG. 25, according to one embodiment.

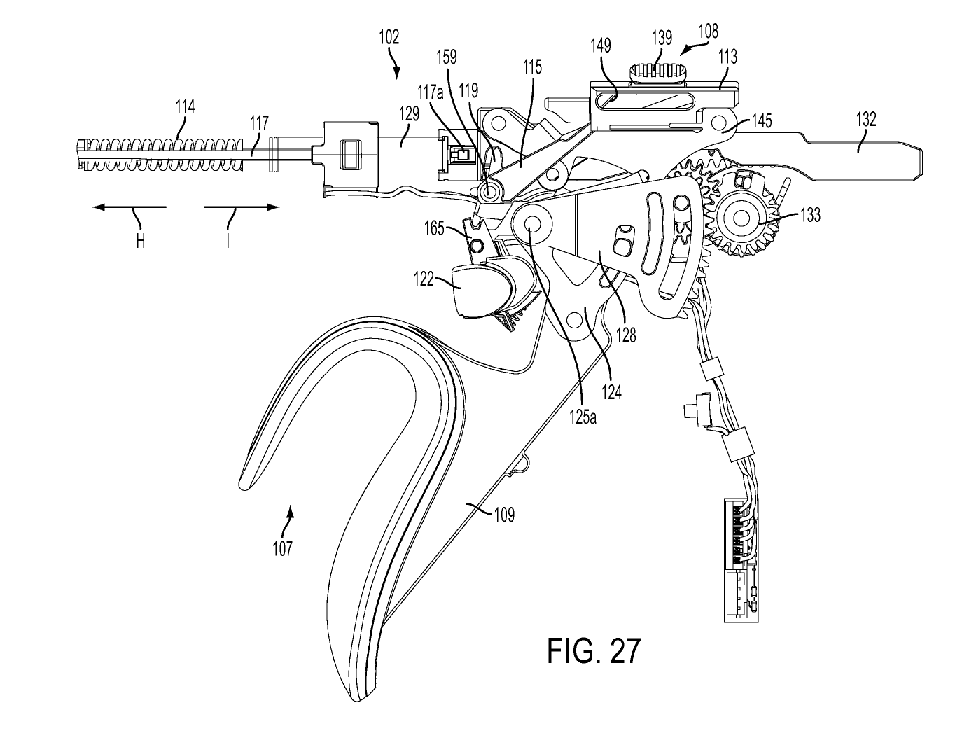

FIG. 27 is a side elevational view of the surgical instrument 102 shown in FIG. 24 with the right housing shroud 106b removed, according to one embodiment. The trigger 109 is located in the maximum distal position and the trigger plate 124 is engaged with the toggle clamp 145 and yoke 132, which are located in the maximum proximal position to set the jaws 110 in the fully open position. The slider 113 is set to the maximum proximal “B” position where the angled wall (ramp) 149 has rotated the lever arm 115. The lever arm 115 rotates the unlock arm 119 clockwise and the lockout element 165 counterclockwise to enable the lockout defeat mechanism 108. The lockout element 165 also depresses the energy button 122 to indicate that the lockout defeat mechanism 108 enabled in the “ON” position. This view also shows the position of the firing plate 128 sector gear meshed with the first pinion 133 prior to firing the cutting element. In this configuration the jaws 116a, 116b can be fully closed independently of the firing bar 117 driving the I-beam member 216 by squeezing the trigger 109 to drive the toggle clamp 145 and the closure spring 114.

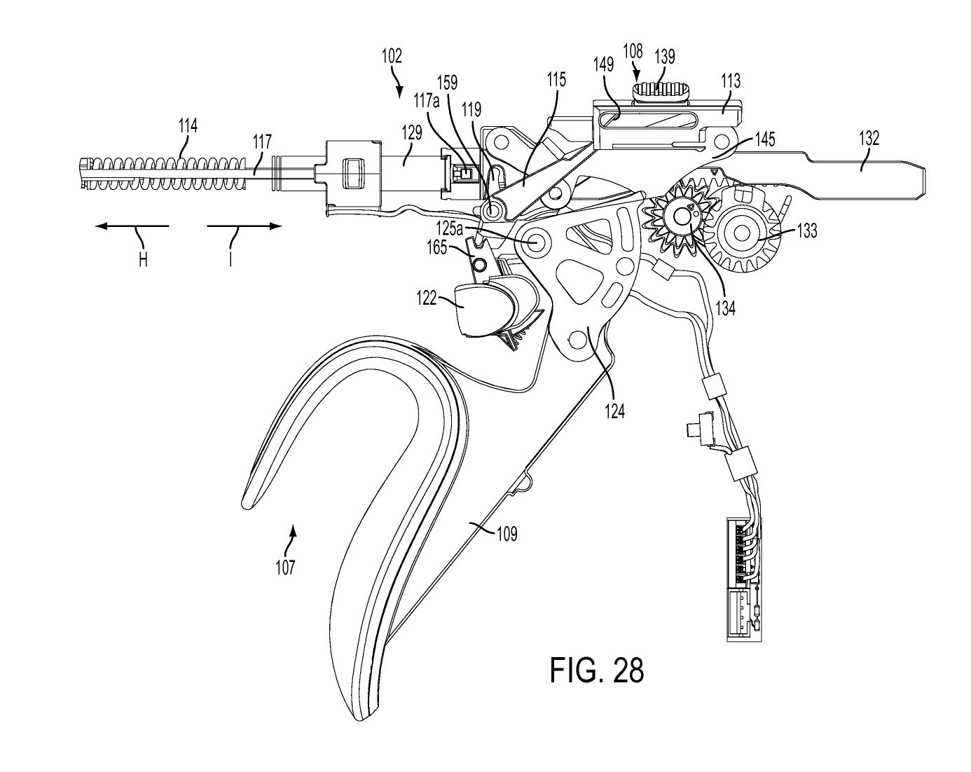

FIG. 28 is a side elevational view of the surgical instrument shown in FIG. 27 with the firing plate 128 removed, according to one embodiment. This view illustrates the position of the trigger 109 relative to the trigger plate 124, the toggle clamp 145, and the yoke 132. This view also shows the first pinion 133 meshed with the second pinion 134 which located behind the firing plate 128.

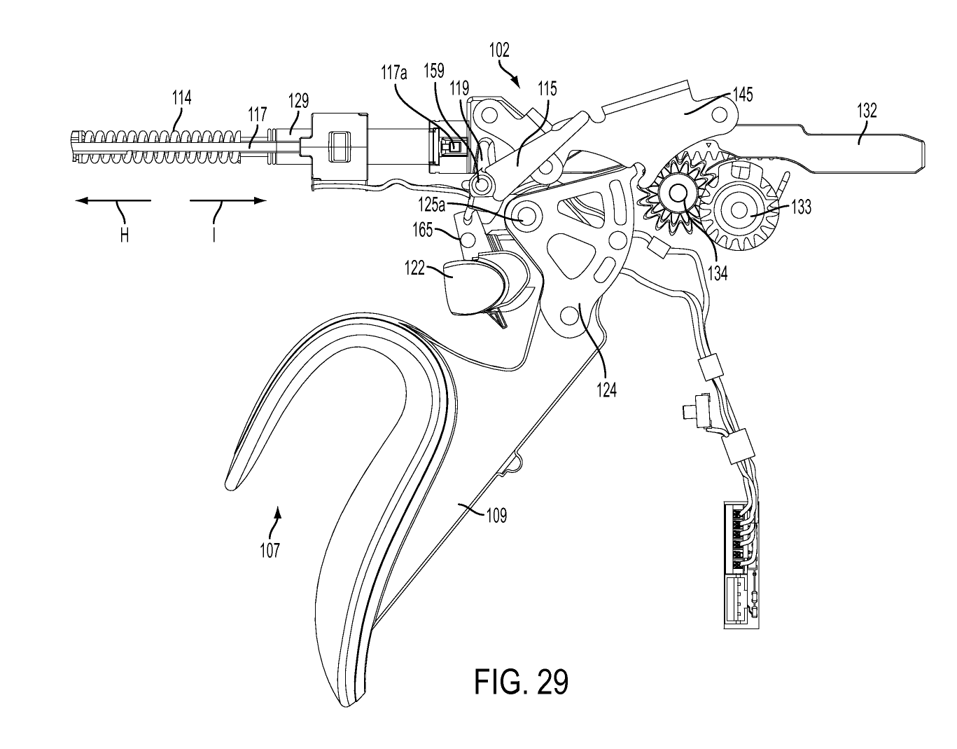

FIG. 29 is a side elevational view of the surgical instrument 102 shown in FIG. 28 with the lockout defeat mechanism slider 113 removed, according to one embodiment, to better illustrate the position of the toggle clamp 145 when the jaws 110 are fully open.

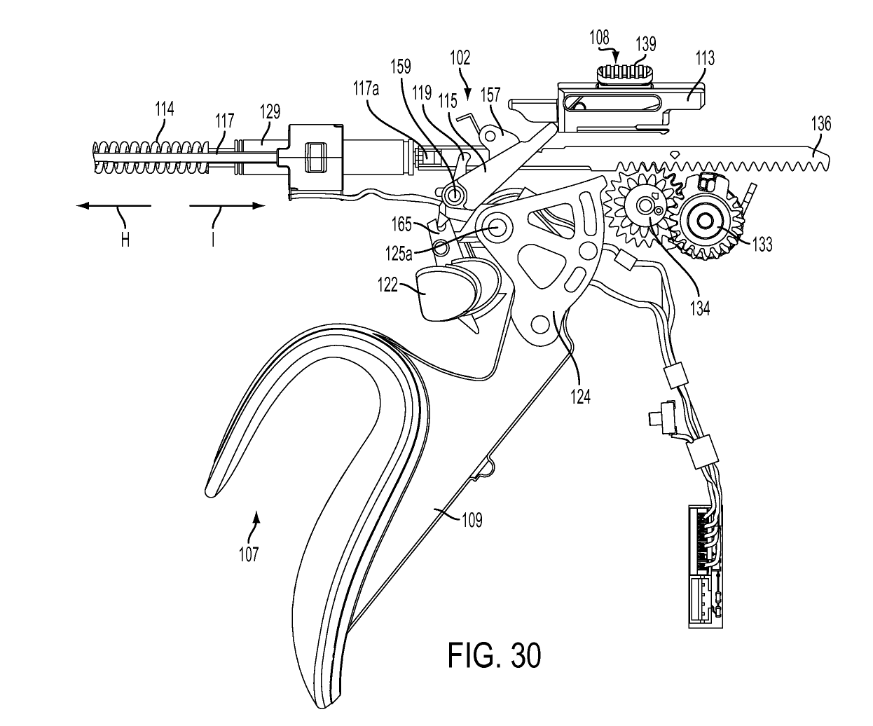

FIG. 30 is a side elevational view of the surgical instrument 102 shown in FIG. 29 with the toggle clamp 145 and the yoke 132 removed, according to one embodiment. This view shows the position of the rack 136 and the lock arm 157 relative to the position of the slider 113. In addition, this view shows the second pinion 134 meshed with the rack 136 when the cutting element has not yet been fired.

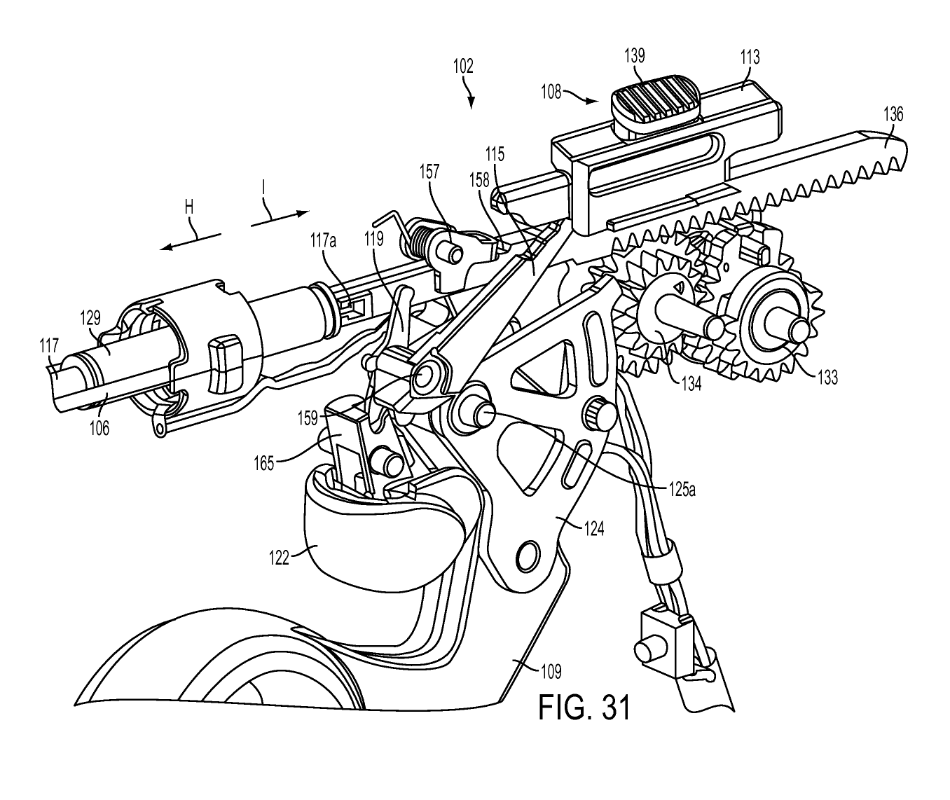

FIG. 31 is a partial perspective view of the surgical instrument 102 shown in FIG. 30, according to one embodiment, which more clearly shows the lock arm 157 located in the notch 158 formed on top of the rack 136. When the unlock arm 119 is in the indicated position, as the toggle clamp 145 and yoke move in the distal direction, the unlock arm 119 acts on the lock arm 157 to disengage the lock arm 157 from the notch 158 in the rack 136 to defeat the lockout mechanism. Therefore, the rack 136 is able to advance distally when the firing plate 128 is rotated by the trigger 109.

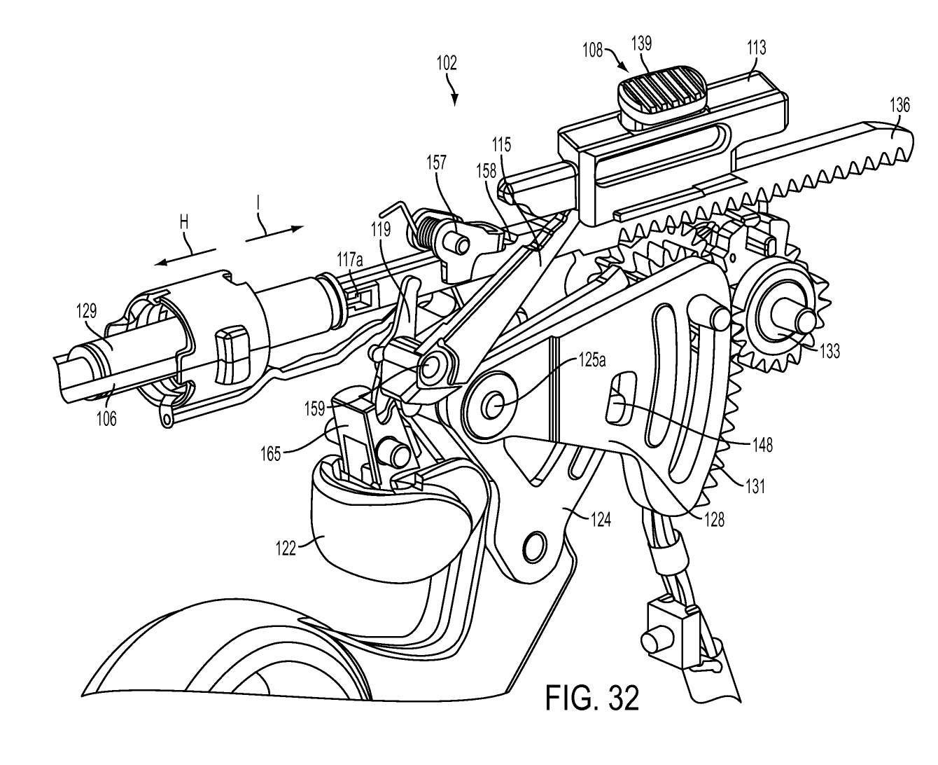

FIG. 32 is a partial perspective view of the surgical instrument shown in FIG. 31 with the firing plate 128 replaced, according to one embodiment, to show the relative position of the firing plate 128, the first and second pinions 133, 134 and the rack 136 prior to firing the cutting element.

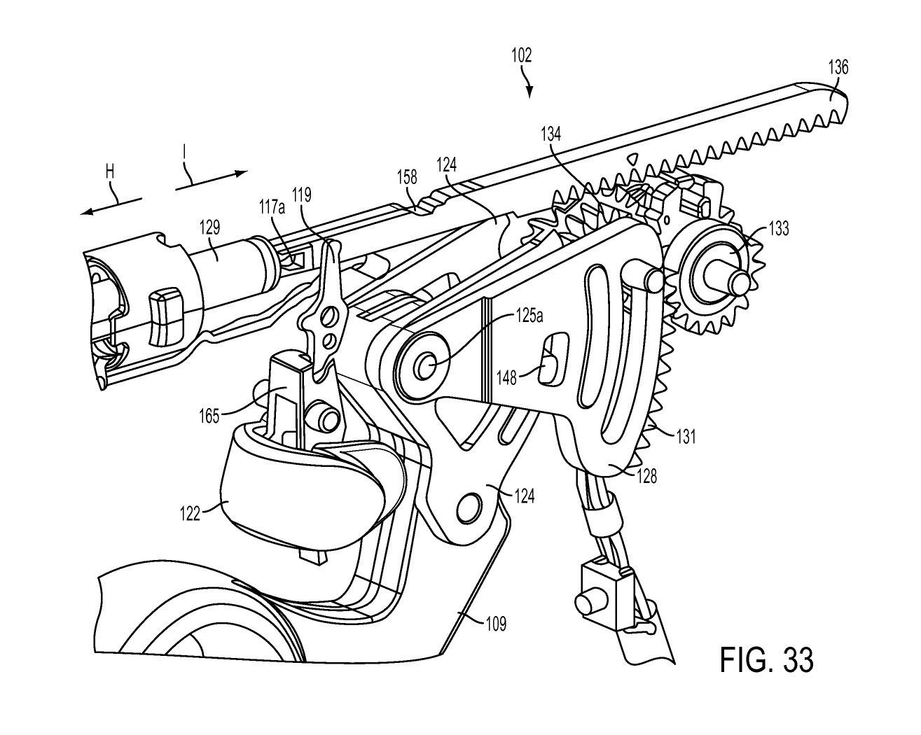

FIG. 33 is a partial perspective view of the surgical instrument 102 shown in FIG. 32 with the lockout defeat mechanism slider 113, lever arm 115, and lock arm 157 removed, according to one embodiment, to show the notch 158 or slot formed on top of the rack 136. As previously discussed, the lock arm 157 engages the notch 158 to prevent the rack 136 from advancing distally to fire the cutting element in response to the squeezing the trigger 109.

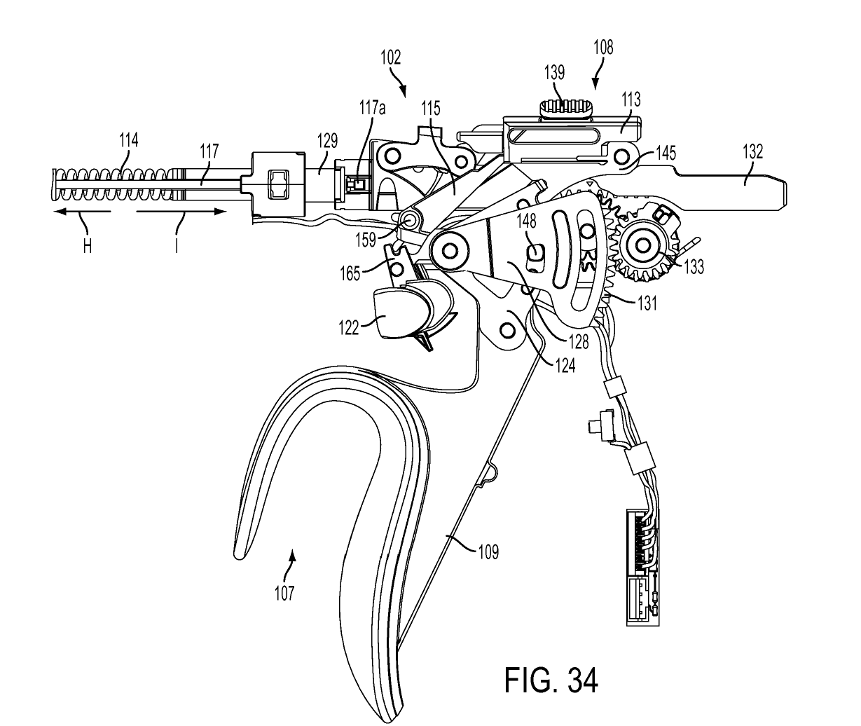

FIG. 34 is a side elevational view of the surgical instrument 102 shown in FIGS. 1 and 2 with the left and right housing shrouds 106a, 106b removed, shaft assembly 112 sheaths removed, the jaws 116a, 116b clamped and the lockout defeat mechanism 108 enabled, e.g., in the “ON” position, according to one embodiment. The trigger plate 124 is fully rotated counterclockwise to straighten the toggle clamp 145 and drive the yoke 132 distally in direction H. To fully close the jaw 110, the trigger 109 is squeezed in direction C to rotate the trigger plate 124 fully counterclockwise to straighten the toggle clamp 145 and advanced the yoke 132. Since the knife 174 has not been fired, the trigger 109 has not been fully squeezed and the firing plate 128 has not been rotated to actuate the rack 136. The yoke 132 is coupled to the closure actuator 129 which compresses the closure spring 114 and drives the closure bar 142. The closure bar 142 is coupled to the pivoting link 178a which closes the upper jaw 116a.

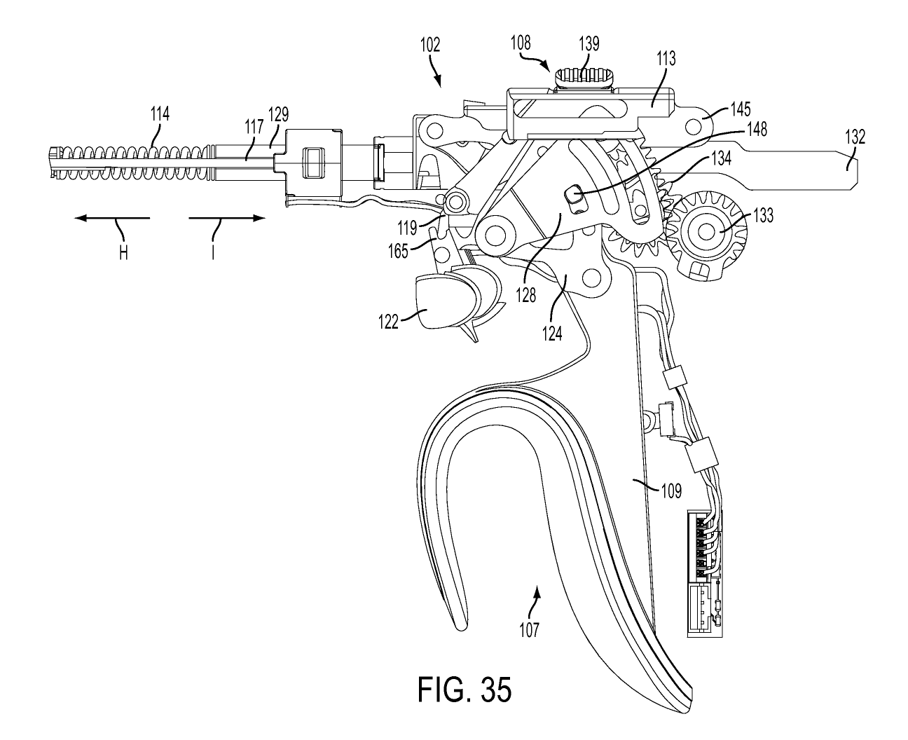

FIG. 35 is a side elevational view of the surgical instrument 102 shown in FIGS. 1 and 2 with the left and right housing shrouds 106a, 106b removed, shaft assembly 112 sheaths removed, jaws 116a, 116b fully closed, knife 174 fully fired, and the lockout defeat mechanism 108 disabled, e.g., in the “OFF” position, according to one embodiment. The button 139 portion of the slider 113 is slidably moved distally to locate it in the A position. To fully close the jaw 110, the trigger 109 is squeezed in direction C to rotate the trigger plate 124 fully counterclockwise to straighten the toggle clamp 145 and advanced the yoke 132. As indicated by the position of the trigger 109 and the firing plate 128, the knife 174 is fully fired.

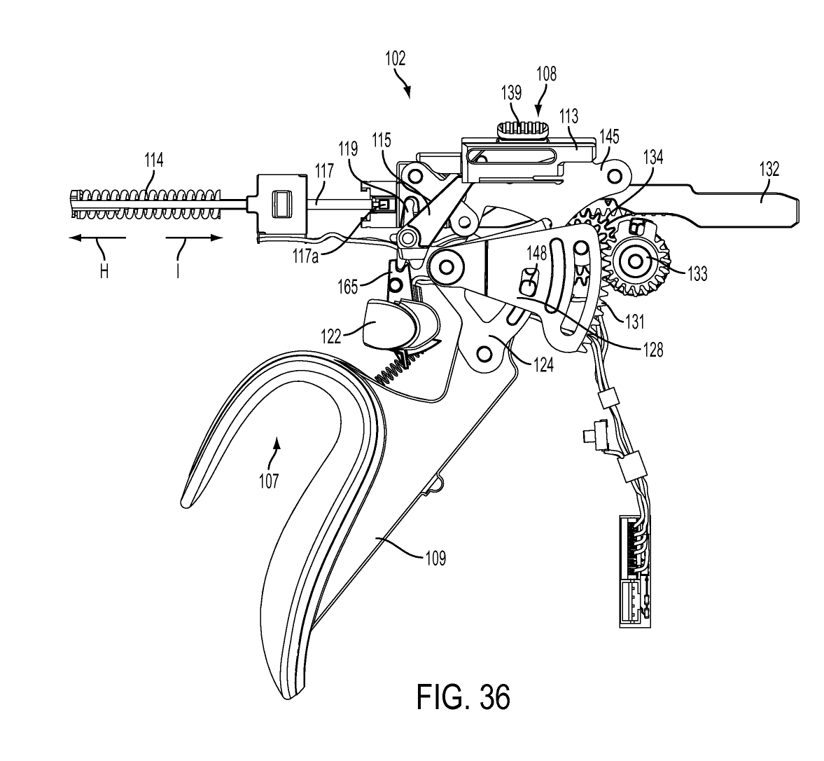

FIG. 36 is a side elevational view of the surgical instrument 102 shown in FIGS. 1 and 2 with the left and right housing shrouds 106a, 106b removed, shaft assembly 112 sheaths removed, jaws 116a,116b fully open, knife 174 not fired, and the lockout defeat mechanism disabled 108, e.g., in the “OFF” position, according to one embodiment. To fully fire the knife while the lockout defeat mechanism 108 disabled, e.g., in the “OFF” position (in other words, the lockout mechanism is enabled) the energy button 122 must be depressed to rotate the lockout element 165 counterclockwise and rotate the unlock arm 119 clockwise to kick the lock arm 157 out of the notch 158 in the rack 136 and unlock the lockout mechanism. Once the lockout mechanism in unlocked, the trigger 109 can be fully squeezed in direction C to rotate the firing plate 128 counterclockwise. This rotates the first pinion 133 clockwise, the second pinion 134 counterclockwise, and the rack 136 is driven distally to fire the firing bar 117 distally in direction H to fire the knife 174 and the I-beam member 216.

It is worthy to note that any reference to “one aspect,” “an aspect,” “one embodiment,” or “an embodiment” means that a particular feature, structure, or characteristic described in connection with the aspect is included in at least one aspect. Thus, appearances of the phrases “in one aspect,” “in an aspect,” “in one embodiment,” or “in an embodiment” in various places throughout the specification are not necessarily all referring to the same aspect. Furthermore, the particular features, structures or characteristics may be combined in any suitable manner in one or more aspects.

Although various embodiments have been described herein, many modifications, variations, substitutions, changes, and equivalents to those embodiments may be implemented and will occur to those skilled in the art. Also, where materials are disclosed for certain components, other materials may be used. It is therefore to be understood that the foregoing description and the appended claims are intended to cover all such modifications and variations as falling within the scope of the disclosed embodiments. The following claims are intended to cover all such modification and variations.

Although various embodiments have been described herein, many modifications, variations, substitutions, changes, and equivalents to those embodiments may be implemented and will occur to those skilled in the art. Also, where materials are disclosed for certain components, other materials may be used. It is therefore to be understood that the foregoing description and the appended claims are intended to cover all such modifications and variations as falling within the scope of the disclosed embodiments. The following claims are intended to cover all such modification and variations.

1. An electrosurgical instrument, the instrument comprising:
a shaft;
an end effector positioned at a distal end of the shaft, the end effector comprising:
a first jaw, the first jaw comprising:
a first jaw body;
a first jaw energy delivery surface;
a first jaw electrode positioned at the first jaw energy delivery surface;
an insulator positioned between the first jaw electrode and the first jaw body to thermally insulate the first jaw electrode and the first jaw body; and
an inner surface positioned at the first jaw energy delivery surface, thermally coupled to the first jaw body, and electrically coupled to the first jaw body; and
a second jaw pivotable towards the first jaw from an open position to a closed position, wherein the second jaw comprises a second jaw energy delivery surface, and wherein the first jaw energy delivery surface and the second jaw energy delivery surface face one another when the end effector is in the closed position.

2. The electrosurgical instrument of claim 1, wherein the second jaw further comprises:
a second jaw body;
a second jaw electrode at the second jaw energy delivery surface; and
a second jaw insulator positioned between the second jaw electrode and the second jaw body to thermally insulate the second jaw electrode and the second jaw body.

3. The electrosurgical instrument of claim 2, wherein the first jaw comprises a positive temperature coefficient (PTC) element at the first jaw energy delivery surface.

4. The electrosurgical instrument of claim 3, wherein the first jaw electrode and the PTC component face the second jaw electrode, wherein the inner surface comprises a tooth having a surface opposite at least a portion of the second jaw electrode, and wherein a width of the second jaw electrode is about equal to a sum of: a width of the first jaw electrode; a width of the at least one surface of the tooth; and a width of the PTC component.

5. The electrosurgical instrument of claim 4, wherein the width of the first jaw electrode is between about 39% and about 45% of the width of the second jaw electrode.

6. The electrosurgical instrument of claim 4, wherein the width of the PTC component is between about 33% and 39% of the width of the second jaw electrode.

7. The electrosurgical instrument of claim 4, wherein the width of the at least one surface is between about 19% and 25% of the width of the second jaw electrode.

8. The electrosurgical instrument of claim 2, wherein the first jaw electrode faces the second jaw electrode, wherein the inner surface comprises a tooth with at least one surface opposite at least a portion of the second jaw electrode, and wherein a width of the second jaw electrode is about equal to a sum of: a width of the first jaw electrode and a width of the at least one surface of the inner surface.

9. The electrosurgical instrument of claim 1, wherein the first jaw electrode is coupled to the first jaw body at least one connection point, wherein the at least one connection point is positioned at a proximal portion of the first jaw body.

10. The electrosurgical instrument of claim 1, wherein the first jaw defines a first jaw channel, the second jaw defines a second jaw channel, and further comprising a cutting element extendable distally through the first and second jaw channels to transition the end effector to the closed position.

11. The electrosurgical instrument of claim 10, wherein the inner surface is positioned adjacent the first jaw channel, wherein the inner surface comprises a plurality of teeth, and wherein a first portion of the plurality of teeth are positioned on a first side of the first jaw channel and a second portion of the plurality of teeth are positioned on a second side of the first jaw channel.

12. The electrosurgical instrument of claim 1, wherein the inner surface comprises a plurality of teeth positioned at a regular interval along a length of the first jaw.

13. The electrosurgical instrument of claim 12, wherein the inner surface further comprises inter-teeth recesses positioned between the plurality of teeth.

14. An electrosurgical instrument, the instrument comprising:
a shaft;
an end effector positioned at a distal end of the shaft, the end effector comprising:
a first jaw, the first jaw comprising:
a first jaw body; and
a first jaw energy delivery surface; and
a second jaw pivotable towards the first jaw from an open position to a closed position, wherein the second jaw comprises:
a second jaw body;
a second jaw energy delivery surface, wherein the first jaw energy delivery surface and the second jaw energy delivery surface face one another when the end effector is in the closed position;
a second jaw electrode positioned at the second jaw energy delivery surface;
a second jaw inner surface positioned at the second jaw energy delivery surface and thermally connected to the second jaw body; and
a second jaw insulator positioned between the second jaw heat conductor and the second jaw body.

15. The electrosurgical instrument of claim 14, wherein the inner surface comprises a plurality of teeth.

16. The electrosurgical instrument of claim 15, wherein the first jaw defines a first jaw channel, the second jaw defines a second jaw channel, and further comprising a cutting element extendable distally through the first and second jaw channels to transition the end effector to the closed position.

17. The electrosurgical instrument of claim 16, wherein the plurality of teeth comprises a first tooth on a first side of the second jaw channel and a second tooth on a second side of the second jaw channel opposite the first heat conductor.

18. The electrosurgical instrument of claim 16, wherein the first jaw further comprises a first jaw electrode positioned at the first jaw energy delivery surface and an insulator positioned between the first jaw electrode and the first jaw body to thermally insulate the first jaw electrode and the first jaw body.

19. The electrosurgical instrument of claim 18, wherein the first jaw further comprises a first jaw inner surface comprising a plurality of teeth and a plurality of inter-teeth recesses positioned between the plurality of teeth, and wherein when the end effector is in the closed position, the second jaw heat conductor aligns with at least one of the inter-teeth recesses.

20. The electrosurgical instrument of claim 14, wherein the second jaw insulator is also positioned between the second jaw electrode and the second jaw body to thermally insulate the second jaw electrode from the second jaw body.
